(19) United States
(12) Patent Application Publication
(10) Pub. No.: US 2009/0155838 A1
(43) Pub. Date: Jun. 18, 2009

(54) Devices, systems and methods for the collection, stimulation, stabilization, and analysis of a biological sample

(75) Inventor: Matthew Hale, Palo Alto, CA (US)

Correspondence Address: WILSON SONSINI GOODRICH & ROSATI, 650 PAGE MILL ROAD, PALO ALTO, CA, 94304-1050, US

(73) Assignee: Smart Tube, Inc., Palo Alto, CA (US)

(21) Appl. No.: 12/315,186

(22) Filed: Nov. 28, 2008

Related U.S. Application Data
(60) Provisional application No. 60/990,626, filed on Nov. 28, 2007.

(51) Int. Cl. C12Q 1/02; C12M 1/00; C12M 1/34; G01N 33/48; G01N 33/53; C12Q 1/68; C40B 30/04; A01N 1/02; G01N 27/26

(52) U.S. Cl. 435/29; 435/287.1; 435/287.2; 435/286.1; 435/40.5; 435/7.1; 435/6; 506/9; 435/7.2; 435/2; 205/792

(57) ABSTRACT

Devices, systems, methods and kits for the collection, stimulation, stabilization and analysis of biological samples, including blood samples, are disclosed. An embodiment of the invention includes a container having a side wall, a bottom wall and a closure member defining an internal compartment having arranged therein a partition defining and fluidly separating first and second chambers in the internal compartment, the first chamber positioned in association with the closure member to receive the biological sample; in which at least one wall is constructed of an elastically deformable material; in which the first chamber contains at least one stimulating agent; in which the second chamber contains at least one stabilizing agent; and in which the first and second chambers can be placed in fluid communication by a user without opening or otherwise compromising the fluid integrity of the internal compartment.

### CROSS-REFERENCE

This application claims the benefit of U.S. Provisional Application No. 60/990,626, filed Nov. 28, 2007, which application is incorporated herein by reference in its entirety.

### BACKGROUND OF THE INVENTION

Conventional diagnostics have focused on measuring the unperturbed biological state of a sample. In recent years there has been growing interest in exposing patient material to stimulatory agents such as cytokines, immunomodulatory factors, existing drugs, and new drug candidates, and then measuring the changes that have been induced in numerous cellular parameters such as intracellular signal transduction and genome-wide transcription. Several studies have shown that interrogating patient samples with stimuli reveals otherwise invisible biological states that have substantial clinical and diagnostic value (Irish, J. M. et al. Single cell profiling of potentiated phospho-protein networks in cancer cells. Cell (2004) 118, 217-28; Van Meter, M. E. et al. K-RasG12D expression induces hyperproliferation and aberrant signaling in primary hematopoietic stem/progenitor cells. Blood (2007) 109, 3945-52).

A significant obstacle is that the majority of facilities that routinely draw blood lack the ability to carry out well-controlled stimulation experiments. Specific problems include preparation of the stimulus, delivery of a precise amount of stimulus to the blood sample, and stabilizing the sample for later assessment of signaling state or transcript abundance. Many of these facilities lack the equipment necessary to carry out conventional stimulation experiments. Currently, live patient samples are shipped to laboratories capable of carrying out the assays of interest or are cryopreserved prior to shipping. Unfortunately, it is undesirable to ship certain samples in an unfixed/unstabilized state including blood samples positive for HIV or other infectious agents, and proper cryopreservation is also beyond the capabilities of many facilities. Moreover, both cryopreservation and live shipping have been shown to induce changes in intracellular signaling and gene transcription and yield results that have been shown to poorly reflect the biology of blood cells in their native context.

Sample collection containers have been in use for many years for collecting and storing blood and other body fluids. Typically, the collection containers are glass or plastic having a resilient stopper. Blood collection tubes are available where the tube is evacuated to draw a defined volume of blood into the tube. The tubes can have various additives contained therein for preparing the blood sample for a particular test. A common additive is an anticoagulant such as ethylenediaminetetraacetic acid (EDTA), buffered citrate, or heparin. Other tubes contain one or more fixatives that stabilize the nucleic acids in the sample. Such agents can be present in liquid or dried state. These existing sample collection containers are not capable of executing multi-step experiments unless the user employs liquid handling devices that are not available at most locations where blood is drawn. Stimulation experiments require a minimum of two separate steps that must be carefully timed. In the first step the sample is treated with an anticoagulant and exposed to stimuli. After a defined period of time the second step is to add a stabilizing solution that freezes and preserves the proteomic and or genomic character of the cell for storage, shipment, and later analysis.

Therefore, it would be desirable to have a means for collecting and stimulating biological samples, such as, e.g., blood samples, for subsequent analysis, where the stimulation experiments have high precision and consistency. Herein are disclosed devices, systems, methods, and kits to accomplish these and other aims.

### SUMMARY OF THE INVENTION

Devices, systems, methods and kits for the collection, stimulation, stabilization and analysis of biological samples, including blood samples, are provided herein. To obtain the most physiologically and clinically relevant results from stimulation experiments performed on a biological sample, the biological sample should ideally be stimulated with a controlled dose of stimulus immediately after being obtained from a patient and, after a defined time interval of stimulation, the resulting intracellular signaling and/or gene transcription rapidly frozen in state by one or more stabilizing agents.

An apparatus for collecting, assaying and stabilizing a biological sample is provided herein. In some embodiments, the apparatus includes a container having a side wall, a bottom wall and a closure member defining an internal compartment, in which at least one wall is constructed of an elastically deformable material.

In one aspect, the internal compartment has, arranged inside, a partition which defines and fluidly separates first and second chambers within the internal compartment.

In one aspect, the first chamber is positioned in association with the closure member to receive the biological sample. In a further aspect, the first chamber contains at least one stimulating agent. A stimulating agent, or stimulus as referred to herein, can include any agent, such as, e.g., a biological agent, placed in the first chamber which results or has the potential to result in a biological change in the biological sample.

In another aspect, the second chamber contains at least one stabilizing agent. A stabilizing agent, as referred to herein, can include any agent which maintains in state, i.e., inhibits any further change in, the status of any biomolecule in the biological sample.

In one aspect, the first and second chambers can be placed in fluid communication by deforming a wall of the container without opening the internal compartment of the container or otherwise compromising the fluid integrity of the internal compartment.

In some embodiments, the partition is constructed of a material the fluid integrity of which can be compromised by deformation of a wall so as to place the first and second chambers in fluid communication.

In some embodiments, an elastically deformable wall further includes a support structure, such as a support ring, at the interior of the internal compartment, such that the partition includes a disc member affixed by a breakable adhesive to the support ring, the affixed disc member defining and fluidly separating the first and second chambers in the internal compartment; in which the disc member is constructed of a material substantially less elastically deformable than the wall such that the disc member can be displaced by deformation of the wall and support ring or structure, so as to place in fluid communication the first and second chambers.

Also provided herein are systems for collecting, assaying and stabilizing a biological sample. In one aspect, the systems include a collection apparatus as described above and additionally, an automation apparatus, also referred to herein as a base station, which automates certain aspects of using the collection apparatus and can facilitate the use of multiple collection apparati in parallel to stimulate, stabilize, and store multiple biological samples, as well as to store and track information regarding each use. In one aspect, the automation apparatus includes a manipulation means which is capable of manipulating the collection apparatus. In a second aspect, the automation apparatus includes a force-exerting means capable of placing in fluid communication the first and second chambers of the collection apparatus. In a third aspect, the automation apparatus includes a thermal regulation means capable of regulating the temperature of the collection apparatus. In some embodiments, the system provided herein further includes a microelectronic element controlling the functions of the automation apparatus. In some embodiments, the automation apparatus provided herein further includes a timing means in functional communication with, and arranged so as to trigger the operation of one or more of: the manipulation means, the force-exerting means and the thermal regulation means.

In some embodiments of the system, the collection apparatus further includes a unique tag allowing its identification. In another aspect of the system, the automation apparatus further includes the means to scan and identify each tagged collection apparatus, as well as a database capable of storing assay parameter data for one or more uniquely tagged collection apparati.

Also provided herein are methods of collecting, stimulating and stabilizing a biological sample using the described apparatus. In a further aspect, the methods disclosed herein include providing an automation apparatus as described above, in which the apparatus automatically performs the steps of stimulating, stabilizing and storing the biological sample. Some embodiments of the herein disclosed methods further include analyzing the sample by proteomic or genomic methods.

Kits for collecting, assaying and stabilizing a biological sample according to the herein described methods are also provided.

These and other objects, advantages, and features of the invention will become apparent to those persons skilled in the art upon reading the details of the invention as more fully described below.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a side view of one embodiment of the container apparatus (Smart Tube); FIG. 1B is a lateral cross-section of the device shown in FIG. 1A; FIG. 1C is a bottom view of the device shown in FIG. 1A; FIG. 1D is an exploded view of the device. FIG. 1E is an exploded perspective view of the device. FIG. 1F is an enlarged top view of the ampoule retention insert with its aperture visible. FIG. 1G is a lateral cross-section of the ampoule retention insert with the aperture geometry visible. FIG. 1H is a perspective view of the ampoule retention insert with the top of the insert visible. FIG. 1I is a perspective view of the ampoule retention insert with the bottom of the insert visible;

FIG. 2 shows front (FIG. 2A), top (FIG. 2B), and front cross-section (FIG. 2C) views of the prototype configuration of the apparatus. Included are dimensions of the prototype container, in inches;

FIG. 3A is a front view of tube made of flexible and resilient material FIG. 3B is a cross section view of tube showing the sample collection chamber, hard plastic disc separating the two compartments, and integral support ring to which the disc is attached by breakable adhesive. FIG. 3C is an exploded front view of the tube design showing the oval hard plastic disc. FIG. 3D is an exploded side view of the new tube design showing the oval hard plastic disc. FIG. 3E is a perspective view of the new tube design showing the oval hard plastic disc. FIG. 3F is a perspective view of cross section with disc removed showing the integral support ring. FIG. 3G is a perspective view of cross section with disc removed showing the support ring.

FIG. 4A shows a perspective view of one embodiment of an apparatus; FIG. 4B shows a perspective view of the apparatus in FIG. 4A with the top, left, and front panels removed along with the two top frame members. FIG. 4C illustrates a front view of one embodiment of the apparatus; FIG. 4D illustrates a left side view of one embodiment of the apparatus; FIG. 4E illustrates a top view of one embodiment of the apparatus. FIG. 4F illustrates a front view of one embodiment of the apparatus with the front panel removed. FIG. 4G illustrates a left side view of one embodiment of the apparatus with the left and front panels removed;

FIG. 5A illustrates the armature of the base station automated device in the open position. FIG. 5B illustrates the armature of the base station in the closed position;

FIG. 6A is a side view of the base station automated device. The plane of the cross section in FIG. 6B is shown as a dotted line. FIG. 6B is the cross section of FIG. 1A. The plane of the cross section bisects the tube in the tube block. The thick line square shows the region that is enlarged in FIG. 6C. FIG. 6C is an enlarged view of the region specified by the thick line in FIG. 6B and shows a bisected tube in the tube block. Also shown is the tube interfacing with one of the five couplings that generates axial rotation;

FIG. 7A shows a top view of tube block sub-assembly of the base station automated device with one tube in it. FIG. 7B shows a front view of the tube block sub-assembly with one tube in it. FIG. 7C shows a side view of the tube block sub-assembly with one tube; FIG. 7D shows an exploded perspective view of the tube block sub-assembly with one tube;

FIG. 8A shows an exploded top view of the tube block sub-assembly of the base station automated device with one tube. FIG. 8B shows an exploded left view of the tube block sub-assembly with one tube. FIG. 8C shows an exploded bottom view of the tube block sub-assembly with one tube;

FIG. 9A shows a perspective view of the tube block and liquid cooling system of the base station automated device with other components removed for clarity. FIG. 9B shows a perspective view of the tube block and liquid cooling system with other components removed for clarity; and

FIG. 10A is a side view of the filter cap suitable for replacing the closure member on the collection apparatus. FIG. 10B is a lateral cross-section of the device shown in FIG. 10A showing the threads that engage the threads on the device (Smart Tube). FIG. 10C is a perspective view of the device shown in FIG. 10A with the top of the device visible. FIG. 10D is a top view of the device shown in FIG. 10A. FIG. 10E is a bottom view of the device shown in FIG. 10A. FIG. 10F is a perspective view of the device shown in FIG. 10A with the bottom of the device visible.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the compound” includes reference to one or more compounds and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

An apparatus for collecting, assaying and stabilizing a biological sample is provided herein. In some embodiments, the apparatus includes a container having a side wall, a bottom wall and a closure member defining an internal compartment, in which at least one wall is constructed of an elastically deformable material. In other words, the internal compartment is formed by the juncture of the side wall, bottom wall and closure member. In some embodiments, any of the side wall, bottom wall, and closure member may be distinct pieces which are assembled to form the internal compartment, or, alternatively, one or more aspects may be fashioned from a single piece of material, such as, without limitation, as molded plastic or metal. In some embodiments, the closure member is removable and replaceable to reveal an open end of the internal compartment.

Where the container is referred to as having at least one wall constructed of an elastically deformable material, it is meant that the shape of the wall can be deformed by sufficient pressure, such as intentional bending or pressing on the surface of the container, and will on its own return to substantially the same shape.

In some embodiments, the elastically deformable wall is at least one of: the side wall; the bottom wall. In other embodiments, the elastically deformable wall may also be an elastically deformable element such as the closure element, i.e. the plug, cap or stopper, which can be constructed so as to be sufficiently flexible that it can be deformed by force to disrupt the partition and place the first and second chambers in fluid communication. Any element of the apparatus with a surface at the exterior of the apparatus may likewise be constructed to allow deformability to this end.

In other embodiments, a deformable wall or element may be non-elastically deformable, i.e., it may not retain its original shape after the application of force so as to disrupt the partition. In such embodiments, the fluid integrity of the wall or element is nonetheless maintained.

In one embodiment of the invention the apparatus facilitates collecting biological tissue, such as e.g. whole blood, stimulating the contained sample with one or more stimulating agents or stimuli, and then stabilizing the sample for storage and later analysis. This can allow for analysis of blood cell responses to stimulations delivered with high precision and consistency even in remote testing locations. Blood can be added to the device. Alternatively, blood can be directly drawn into the device which can contain an anticoagulant and one or more agents designed to induce a response in the blood cells. The agents can be stimuli. After a defined period of time the device can release a stabilization solution from a second chamber or ampoule that can stabilize the intracellular state of the blood cells including changes that have occurred as a result of exposure to the stimuli, including phosphorylation and or other post-translational modifications of cellular proteins and or mRNA transcript abundance.

One of the end uses of the device can be to carry out diagnostic tests on human patients to improve their medical treatment (e.g., stratify leukemia patients, guide treatment of patients with lupus, etc.). To properly execute these tests, the user must be careful to keep track of the time elapsed since blood was drawn into or manually added to the device and activate the device after the proper amount of time has elapsed. The proper amount of time can be defined by the diagnostic protocol for the test in question (e.g. 15 minutes in the case of assays that have been found to be of value for leukemia patients and lupus patients). In many blood draw locations, such as those of hospitals and clinics, the personnel have work-flow constraints that may make it difficult for them to accurately time and activate devices. To reduce handling errors that could negatively influence the diagnostic utility of the devices and increase their ease of handling, an automation apparatus, also referred to herein as a base station, can be used to automate the timing and activation of devices and provide thermal control and sample mixing.

One embodiment of the invention is that of a disposable device, or tube, for collecting, stimulating, stabilizing and storing a biological sample within a multi-chambered collection device. The present invention further comprises a system including an apparatus that can automate the use of the disposable devices, discussed in further detail below. The apparatus can further ensure proper timing, sample mixing, and thermal control. In one embodiment of the present invention, the blood draw apparatus itself can be capable of executing two separate steps. In the first step, the blood can be drawn into the first chamber, or stimulation chamber, of the tube where it can be exposed to an anticoagulant and/or one or more stimuli. In one aspect, the first chamber is positioned in association with the closure member to receive the biological sample. In other words, the presence of the closure member allows the fluid integrity of the first chamber relative to the exterior of the apparatus. In some embodiments the closure member is a plug such as, for example, a cap, stopper or a pierceable self-sealing plug, or other removable and replaceable element which seals an opening to the exterior in the first chamber through which materials may otherwise be introduced into or removed from the apparatus.

In a further aspect, the first chamber contains at least one stimulating agent. A stimulating agent, or stimulus as referred to herein, can include any agent, such as, e.g., a biological agent, placed in the first chamber which results or has the potential to result in a biological change in the biogical sample. In some embodiments, the mechanism of the biological change is known. In some embodiments, the mechanism of the biological change is unknown. In some embodiments, the stimulating agent is a biologically active molecule or compound suspected or known to have a specific binding partner, such as, for example, a receptor e.g., on the surface of a cell in the biological sample, or an intracellular signalling molecule at the interior of a cell, binding to which produces a biological effect in the cell. In some embodiments, contact with the stimulating agent may produce a change in gene expression in a cell. In some embodiments, a stimulating agent may produce a biological effect by acting as an analog, i.e. by mimicking a ligand to a receptor or other binding partner in the cell. In some embodiments, exposure to the stimulating agent results or has the potential to result in an intracellular change in a cell in the biological sample. In some embodiments, exposure to the stimulating agent results or has the potential to result in a cell-surface molecule change on a cell in the biological sample. In some embodiments, exposure to the stimulating agent results or has the potential to result in an intracellular change in a cell in the biological sample. Stimulating agents include, but are not limited to, small molecules; antibodies and fragments thereof; polypeptides; proteins; receptor ligands; polynucleotides; organic compounds; lipopolysaccharides; cytokines; steroids; cells; genetic agents including, for example, shRNA, siRNA, a virus or genetic material in a liposome; inorganic molecules including salts; and others as known in the art.

In some embodiments, stimulating agents may exclude certain substances, which substances are present in the first chamber so as to sustain mechanical amenability of the biological sample to assay and/or manipulation. Such substances can include, without limitation, anticoagulants, compounds or enzymes which digest, denature or dissociate extracellular matrix, including collagen or other extracellular and structural support materials, as well as DNase or other enzymes that digest nucleic acids that may be found in a biological sample. In embodiments where such substances are present in the first chamber, the stimulating agent can exclude such substances, i.e. be other than such substances, being instead an additional substance which results in a specific biological change in the biological sample, where such change can include, without limitation, change in gene expression, change in cell surface molecule abundance; change in viability, change in cellular import or export of molecules, and the like.

A stabilizing agent, as referred to herein, can include any agent which maintains in state, i.e., inhibits any further change in, the status of any biomolecule in the biological sample. Such can include agents which able to effectively stabilize DNA and RNA including mRNA, tRNA, micro RNA, siRNA, and cRNA. Examples of suitable stabilizing agents for stabilizing and preserving nucleic acids and/or preventing gene induction include cationic compounds, detergents, chaotropic salts, ribonuclease inhibitors, chelating agents and the like, and mixtures thereof. Stabilizing agents for proteins including antigens such as cell surface molecules are well known in the art and include, without limitation, compounds that kill a cell but preserve its protein morphology and/or nucleic acids for an extended period of time. Stabilizing agents can include, for example, cross-linking fixatives, such as paraformaldehyde, or precipitants such as ethanol. Stabilizing agents can act by creating covalent linkages between cellular molecules or by precipitating certain intracellular molecules, or by other means. In some embodiments, the stabilizing agent includes a cell lysis buffer. Cell permeabilization buffers are also well known in the art and can contain detergents which permeabilize the cell membrane so as to allow the passage of probes and stains through the membrane. Examples of detergents used in cell lysis buffers include, without limitation, Tween, Triton X-100, saponin, NP-40 and the like. The concentration of cell lysis and permeabilization agents is adjusted for a given end use. When present at lower concentrations, cell lysis or permeabilization may be suboptimal. At higher concentrations, undesirable cellular disruption may occur. Routine empirical approaches can be carried out to determine the preferred route in each instance.

In some embodiments, the stabilizing agent maintains cell surface antigens while arresting cellular processes. Cellular processes targeted for arrest by the stabilizing agent include, for example, intracellular signalling, protein transport, protein modification, protein synthesis, protein degradation, nucleic acid synthesis, nucleic acid degradation, endocytosis, secretion, phosphorylation, dephosphorylation, ubiquitinization, and methylation.

In one aspect, the first and second chambers can be placed in fluid communication by deforming a wall without opening the internal compartment of the container or otherwise compromising the fluid integrity of the internal compartment. In other words, pressure exerted, by manual or other mechanical means, on the side or bottom wall sufficient to deform the wall results in placement of the first and second chambers, previously separated by the partition, in fluid communication such that the contents of the chambers can mix. This placement in fluid communication of the first and second chambers is a result of disruption of the fluid integrity of the partition.

In some embodiments, the partition is constructed of a material the fluid integrity of which can be compromised by deformation of the compartment wall so as to place the first and second chambers in fluid communication. The capacity of the partition to be so disrupted is in some embodiments due to the material from which it is constructed. The partition can be constructed, for example, of a material that is breakable, in whole or in part, by sufficiently forceful contact with the deformed wall as a result of externally applied pressure. Examples of such materials include, without limitation, plastic or glass, such as borosilicate glass. In some embodiments, the apparatus further includes a mesh or an aperture through which liquid can be added or removed to the internal compartment while retaining within the internal compartment fragments of the compromised partition. Aperture, as used herein, refers to an opening with reticulated edges such that fluid flow through the opening is facilitated by the edge geometry, while the passage of fragments of crushed or broken partition through the opening is inhibited.

In some embodiments, the partition is deformable or elastically deformable, such that deformation of the side or bottom wall results in a physical conformation of the partition which permits fluid communication between the first and second chambers. In some embodiments, the partition is dissolvable. In further embodiments the partition is dissolvable only at a certain temperature. For example, a partition so constructed may be insoluble at room temperature, but become dissoluble when heated to a different temperature such as, for example, 37°, 42° or higher.

In some embodiments, the elastically deformable wall further includes a support ring at the interior of the internal compartment, in which the partition includes a disc member affixed by a breakable adhesive to the support ring, the affixed disc member defining and fluidly separating the first and second chambers in the internal compartment; in which the disc member is constructed of a material substantially less elastically deformable than the wall such that the disc member can be displaced by deformation of the wall and support ring, so as to place in fluid communication the first and second chambers. In some embodiments, the support ring is an integral support ring, such that the ring protrudes from and is composed of the same material as the wall. Where the disc member is referred to as substantially less elastically deformable than the wall, it is meant that deforming the wall by manual or mechanical means will not deform the disc prior to the breaking of the adhesive affixing the disc to the support ring as a result of shear force on the adhesive. In some embodiments, the support ring is at a non-normal angle relative to the long axis of the apparatus. In some embodiments, the angle is an angle which maximizes shear force on the breakable adhesive due to deformation of the side wall, such as e.g., about 45 degrees. By breakable adhesive is meant an adhesive with a known shear strength such that, upon the application of preselected shear force, the adhesive will crack, break, or otherwise be disrupted such that its adhesion function is lost. One of skill in the art can readily identify suitable adhesives for this use.

In still other embodiments, the disc is affixed to the wall in the absence of a support ring and relies on the breakable adhesive to maintain immobility so as to function as a partition.

In further embodiments, instead of a disc, any other solid form, shape or membrane can be interposed within the internal compartment, forming a seal so as to fluidly separate and define the first and second chambers. This solid form can then be dislodged, broken or disrupted by deformation of the wall of the container, as discussed, placing the first and second chambers in fluid communication.

As such, after a defined period of time, the sample can be stabilized in a second step by being mixed with a stabilizing solution from a second chamber or ampoule of the apparatus. Stabilizing the sample can enable storage and later analysis of the sample. These two steps can be executed within the tube and do not require that the stopper be removed or use of any materials other than the standard needle and tubing required to draw blood or otherwise collect the sample of interest (U.S. Pat. No. 2,460,64). The apparatus (Smart Tube) thereby makes it possible for stimulation experiments to be executed in nearly any location where blood is drawn.

An aspect of the invention is to provide a device for collecting a biological sample, and particularly whole blood, from a patient into a chamber containing an anticoagulant and one or more stimuli. The blood sample can be a whole blood. In some embodiments, the blood sample can be plasma. The blood sample can be introduced into the tube. Alternatively, the blood can be drawn directly into the tube. Additionally, the tube can be pre-evacuated to a pressure significantly below that of atmospheric and having a self-sealing rubber stopper. Using standard blood draw tubing blood can be drawn directly from the patient into the tube where it comes into contact with an anticoagulant and stimuli. This embodiment also has a breakable ampoule filled with stabilizer so that at the desired time the stabilizer can be released into the sample and preserve analytes of interest. Thus, this embodiment is a blood collection, stimulation, stabilization, and storage unit.

The stimuli can be agents with known or unknown biological effect on the sample. After a time interval, the device can introduce a volume of fluid containing one or more stabilizing additives into the aforesaid chamber to preserve the intracellular signaling and or transcriptional profile of the patient sample contained therein. The time interval can be user defined. The stabilizing agent can be present in concentrations to effectively arrest intracellular signaling, including post-translational modification of proteins such as phosphorylation, and or gene transcription. The stabilizing agent(s) can also prevent degradation of the analytes of interest and or modification that would interfere with the detection of the analytes of interest. Analytes of interest include, but are not limited to, post-translational modification of proteins including addition or removal of certain chemical groups, such as phosphates, to particular amino acids. Analytes of interest also include, but are not limited to, DNA sequence, messenger RNA sequence, and abundance of messenger RNA transcripts. An agent or agents can be added to the stabilizing fluid to lyse erythrocytes in the sample or facilitate subsequent lysis of erythrocytes.

In some embodiments of the invention, the collection chamber can be evacuated to below atmospheric pressure prior to filling with the sample so as to draw in a pre-determined volume of biological specimen. A pre-determined volume of biological specimen can be especially important for blood specimens. The collection chamber can have an anticoagulant in dried or liquid form in an amount sufficient to prevent coagulation of the specimen.

The objects of the invention can be attained by an apparatus for collecting, stimulating, and stabilizing a biological specimen. In some embodiments, the apparatus is a tube. The tube can be designed to collect biological specimens, or samples, including, but not limited to, blood, synovial fluid, spinal fluid, cerebrospinal fluid, amniotic fluid or tissue biopsies. The body of the tube can comprise of a container comprised of a side wall, a bottom wall, and an open end, defining an internal container, and a closure or stopper closing the open end. The container can be made of any suitable material including, but not limited to, polyethylene, low density polyethylene, linear low density polyethylene, polypropylene, low density polypropylene, nylon, polystyrene, or a combination thereof. The internal container can be the stimulation chamber. In some embodiments, the closure can be a threaded cap made of polyethylene, polypropylene, polystyrene, or any other suitable material or combination thereof.

In some embodiments, a second chamber or ampoule is located inside the container. In some embodiments, the second chamber is located adjacent to the wall of the container. The second chamber can be pre-filled with a stabilizing liquid in an effective amount to stabilize and preserve the biological specimen such that it will preserve the post-translational modifications of cellular proteins. In some embodiments, the stabilizing liquid can preserve phosphorylation and/or halt synthesis and degradation of proteins. While erythrocyte lysis may be desirable where the biological sample is whole blood, in some embodiments of the formulation of the stabilization liquid, the stabilization liquid can prevent lysis of other blood cell types, such as, e.g., leukocytes. The stabilizing liquid can be held separate from the specimen until a period of time after blood draw at which point the stabilizing liquid can then be introduced into the sample. The stabilizing liquid can then stabilize and preserve the biological sample. The amount of time during which the stabilizing liquid can be held separate from the specimen can be user defined.

In one aspect, the internal compartment of the container has, arranged inside, a partition which definines and fluidly separates first and second chambers within the internal compartment. As such, the partition forms one or more walls which separate and prevent the mixture of any contents of the first and second chambers.

In some embodiments, the partition shares structural members with the side wall, bottom wall, and/or closure member; i.e., the partition is, in part or in whole, integral with one or more of the other members forming the internal compartment. In some embodiments, the partition shares no structural members with any of the side wall, bottom wall, or closure member; i.e. the second compartment is defined solely by the partition. In such embodiments the structure of the partition is herein referred to as an ampoule.

In some embodiments of the invention, the stabilizing liquid can be contained in the sealed crushable ampoule or other suitable container, that is held within the stimulation chamber. The stabilizing liquid can be released into the sample when the body of the tube is flexed or bent by being manually or mechanically grasped and bent. When the tube is bent or activated, the inflexible ampoule in the stimulation chamber can be crushed. The stabilizing agent can then be released into the sample.

In some embodiments, the crushable ampoule can be made of thin-walled glass, plastic, fiber, or other suitable material or combination thereof. Mixing of the stabilizing liquid with the sample can then be carried out by shaking or otherwise agitating or vibrating the tube. In some embodiments, the stabilizing liquid can be mixed with the sample by mechanical rotation of the tube. The tube can be rotated along its long axis or along its short axis. In some embodiments, mixing of the stabilizing liquid with the sample can occur by the motion of a magnetic stir bar, or other suitable component, inside the apparatus and acted upon by an external magnetic field or similar force. In some embodiments, ampoule shards are prevented from being mixed with the patient sample by wrapping the ampoule in a closed mesh sheath or bag. The mesh sheath or bag can be made from any suitable biocompatible material including, but not limited to, polypropylene, nylon, or combinations thereof. Alternatively, the ampoule can be coated with any suitable biocompatible material including, but not limited to, silicone rubber, polypropylene, or combination thereof, that will prevent shards of the broken ampoule from being released into the sample. Additionally the shards can be prevented from mixing with the sample by controlling the size that the shards break into. In some embodiments, the shards can be bound together that they will not interfere with downstream processing of the sample. The ampoule can be wrapped, coated, or surface treated with thread or fiber, embedded in silicone rubber or like compound, or coated with a fiber-resin mixture to prevent ampoule shards from being released into the blood or to control the shape of the shards so that they will not interfere with downstream processing of the sample. In alternative embodiments the closure is a self-sealing stopper made of synthetic rubber or like material such as is known in the art.

In an alternate embodiment of the invention, the stabilization liquid can be introduced into the stimulation chamber by electrical, mechanical, or chemical processes. These processes include, but are not limited to, automated mechanical bending of the body of the tube to crush, break or dislodge the partition, such as an ampoule or disc. The stabilizing liquid can then be mixed with the biological sample by means of automated mechanical agitation including rotation, shaking, vibration and the like.

Also provided herein are systems for collecting, assaying and stabilizing a biological sample. In one aspect, the systems include a collection apparatus as described above and additionally, an automation apparatus, also referred to herein as a base station, which automates certain aspects of using the collection apparatus and can facilitate the use of multiple collection apparati in parallel to stimulate, stabilize, and store multiple biological samples, as well as to store and track information regarding each use. In one aspect, the automation apparatus includes a manipulation means which is capable of manipulating the collection apparatus by moving, shaking, rotating, ultrasonically vibrating or subsonically vibrating the collection apparatus, or a combination of such in series.

In a second aspect, the automation apparatus includes a force-exerting means capable of placing in fluid communication the first and second chambers of the collection apparatus. In some embodiments, the force-exerting means exerts pressure upon the elastically deformable wall of the collection apparatus inside so as to disrupt the partition. This can be accomplished by any convenient physical action including striking, bending, pressing upon, twisting the containers, so as to disrupt the partition therein, as described.

In a third aspect, the automation apparatus includes a thermal regulation means capable of regulating the temperature of the collection apparatus. Any technique for regulating temperature may be used, as known in the art.

In some embodiments, the system provided herein further includes a microelectronic element controlling the functions of the automation apparatus. A microelectronic element includes any convenient computational element which, when functionally coupled to the elements of the automation apparatus and provided with an appropriate instruction set, is capable of governing and coordinating the activities of those elements. The skilled artisan will recognize that microprocessors, microcontrollers, embedded controllers, embedded processors, and the like will find use in this aspect of the herein disclosed system.

In some embodiments, the automation apparatus further includes a user interface capable of reporting the status of the system to a user. The user interface can include a light emitting diode (LED), LCD or other kind of display.

In some embodiments, the automation apparatus provided herein further includes a timing means in functional communication with, and arranged so as to trigger the operation of one or more of: the manupulation means, the force-exerting means and the thermal regulation means. The timing means is also functionally linked to the microelectronic element and may be governed by it so as to facilitate the timed functioning of the various elements of the automation apparatus.

As such, the base station can automate certain steps of handling the tube. The base station can hold the sample-containing tube at physiological temperature (37 degrees Celsius) for the duration of the stimulation. The base station can then exert force on the side of the tube to break the ampoule inside. The apparatus can then rotate the tube to mix the stabilizer with the sample, incubate the tube at 37 C for 5 to 10 minutes, then drop the temperature of the tube to a temporary storage temp (5 to 8 degrees Celsius). The tube can then transferred to −80 C for longer storage or dry ice for shipping. In some embodiments, the tube can be manually transferred.

In some embodiments of the system, the collection apparatus further includes a unique tag allowing its identification. Symbolic systems of use in providing a unique tag for each apparatus include a radio frequency identification (RFID) tag, a linear bar code, a matrix or two-dimensional bar code, a microdot pattern and the like as known in the art. In another aspect of the system, the automation apparatus further includes the means to scan and identify each tagged collection apparatus, as well as a database capable of storing assay parameter data for one or more uniquely tagged collection apparati. The automation apparatus in some embodiments further includes a means of transmitting the assay parameter data to a remote location. In some embodiments, the remote location is an external processing system which is capable of one or more of storing, analyzing and displaying the data to a user.

In some embodiments, the tube can have an embedded radio-frequency identification (RFID) tag that can be read by the base station. The Base Station can additionally maintain a database of experimental data linked to each RFID tag. In some embodiments, the base station can keep track of when the tube entered the Base Station, how faithfully the experiment was executed (time of stimulation, thermal profile throughout, measurements of mixing efficiency, whether aberrant electrical phenomenon were detected in the electronics of the Base Station that might indicate inadequate performance), and when the tube was removed from the Base Station. In some embodiments, the database can be accessible by interfacing an external processing system with the Base Station. A variety of different analyses can be performed on the sample following stimulation and stabilization. Of interest in certain embodiments are the analysis protocols described in U.S. Published Application Nos. 20070196870, entitled “Methods and compositions for detecting receptor-ligand interactions in single cells”; 20070009923, entitled titled “Use of Bayesian networks for modeling cell signaling systems”; 20060073474, entitled “Methods and compositions for detecting the activation state, of multiple proteins in single cells”; and 20050112700, entitled “Methods and compositions for risk stratification”, which disclosures are incorporated by reference in their entirety. Alternatively the database can be made accessible by uploading the database onto the network via Ethernet or other connection to a remote server.

### I. DEVICES AND COMPOSITIONS

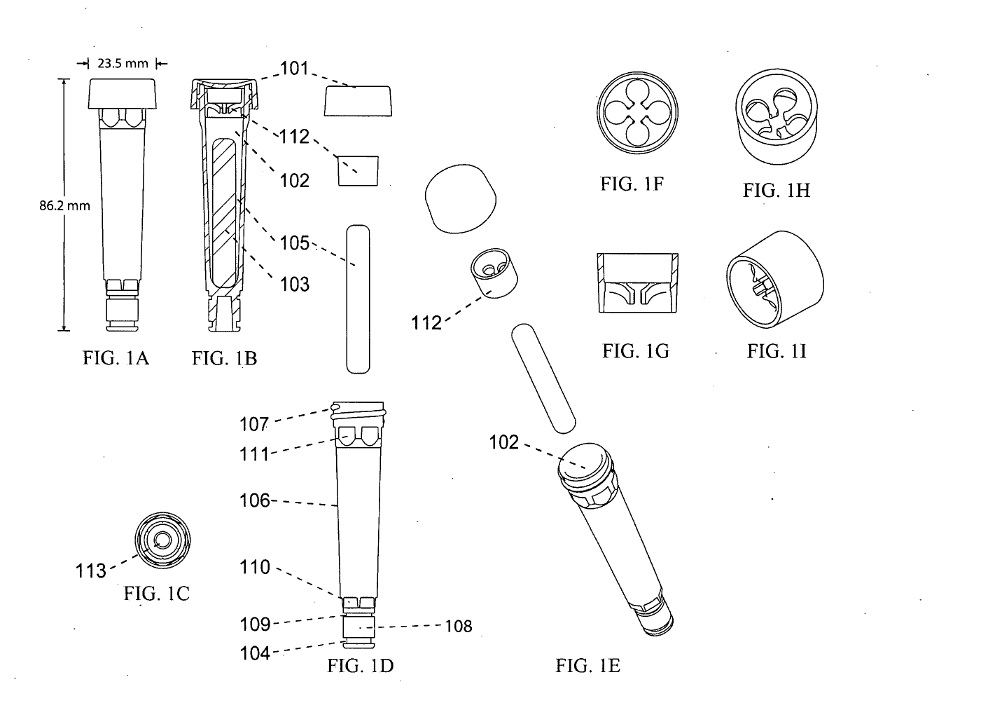

Provided herein is a device suitable for stimulation of a patient sample, especially whole blood, followed by stabilization of the sample in a rapidly-executed second step at the point of collection. FIG. 1 displays several views of one embodiment of a specimen collection tube or container, i.e. the Smart Tube. The body of the device 106 can be made of a flexible resilient, elastically deformable material. Many such materials and methods of working them are known to the art, including e.g. injection molded linear low density polyethylene, and other similarly durable and flexible plastics, fiber composites, metal compositions, and the like. The thin-walled, crushable glass wall 105 of the ampoule defines the ampoule and fluidly separates chambers 102 and 103 until an external force on the flexible wall 106 of the device presses on crushable wall 105 with sufficient force to shatter the crushable wall 105. The contents in the ampoule 103 can then be released into the chamber 102. In some embodiments, 1 milliliter of patient sample can be added to the chamber 102 by a transfer pipette or similar liquid handling device and then the chamber 102 can be sealed shut with a threaded cap 101 interfacing with the threads 107 on the device. The device is designed with a cylindrical region 108 that can be mated with a complementary coupling of an automation apparatus, for example, with a base station. An O-ring can be fitted into groove 104 and can be slightly larger in diameter than the inside diameter of the coupling and is slightly deformed when the cylindrical region 108 is inserted into the coupling of the base station. The device can also have a second groove 109 that allows a retaining clip present in the coupling of the base station to secure the device in the coupling. The coefficient of static friction between the O-ring in 104 and the coupling can allow rotation of the coupling, thereby rotating the device along its long axis (axial rotation). This can facilitate mixing of the contents in the chamber 102. Tapered hexagonal faces at the distal and proximal ends, 110 and 111, respectively, of the device can interface with complementary surfaces on the automation apparatus to apply greater rotational torque to the device to ensure better control of axial rotation. A flexible ampoule retention insert 112 made of LLDPE (linear low density polyethylene) or similar material fitted into the top of the chamber 102 prevents large fragments of the crushed ampoule from leaving the device when its contents are decanted. This insert 112 also prevents the intact ampoule from being removed from 102 accidentally or intentionally, but the downward pointing flexible flanges on the insert allow small pipettes to enter 102 as necessary. A chamber 113 in the bottom of the device 101, is shown in FIG. 1D. The chamber 113 can improve the manufacturability of the device and provides a chamber for the placement of an RFID tag to be added by adhesive or other means. In some embodiments, a biological sample can be drawn into the stimulation chamber 102 by piercing the closure with a hollow needle that is connected to disposable blood draw tubing connected by flexible tubing to another hollow needle already inserted into the patient's vein. The top chamber can be evacuated to a pressure that induces a predetermined volume of blood or fluid, about 1 ml-5 ml, to be drawn in. FIG. 1E shows an exploded view of the device. Chamber 102 can receive a biological sample as well as hold the ampoule.

A sample stabilizing ampoule 103 can be filled with the stabilizing solution in an amount to stabilize a stimulated biological sample received in chamber 102. Stimulating agent is disposed in chamber 102 in an effective amount to stimulate a biological sample received in this chamber.

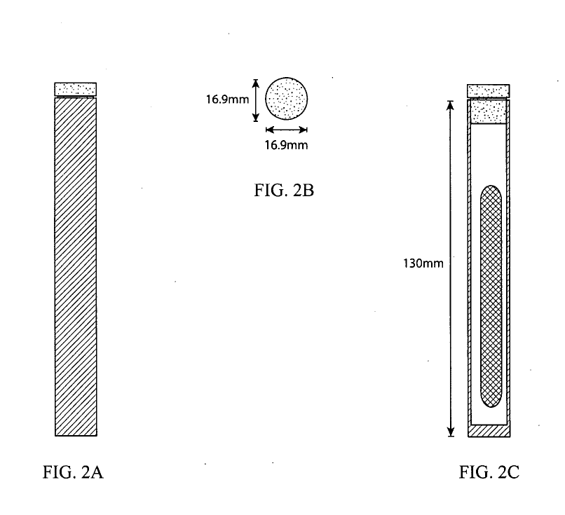

FIG. 2 shows front (FIG. 2A), top (FIG. 2B), and front cross-section (FIG. 2C) views of the original prototype configuration of the apparatus. The flexible body of the container and internally contained ampoule are visible. Included are dimensions of the prototype container, in millimeters.

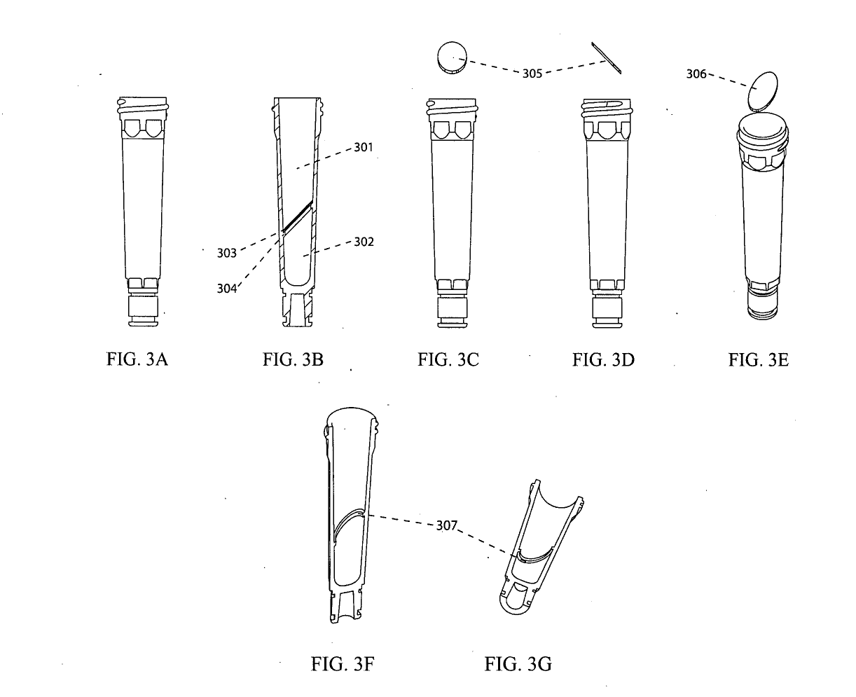

FIG. 3 is an alternate embodiment of a tube, with a disc partition scheme. 3A shows a front view of tube made of flexible and resilient low linear low density polyethylene (or like material) FIG. 3B Cross section view of tube showing the sample collection chamber 301; the chamber containing the stabilizer solution 302; the hard plastic disc 303 that fluidly separates 301 from 302; and the support ring 304 molded as part of the body of the tube to which 303 is attached by releasable adhesive. FIG. 3C Exploded front view of the new tube design showing the oval hard plastic disc 305 that fluidly separates 301 from 302. FIG. 3D Exploded side view of the new tube design showing the oval hard plastic disc 305. FIG. 3E Perspective view of the new tube design showing the oval hard plastic disc 306. FIG. 3F Perspective view of cross section showing support ring 307. FIG. 3G Perspective view of cross section showing support ring 307.

The inside of the first chamber defined or excluded by the partition, stimulation chamber can contain enough lyophilized heparin sulfate to prevent coagulation of the blood sample. The heparin can be previously added to the tube and dried down or lyophilized as known in the art. The stimulus of interest can also be present in the top chamber in dried or lyophilized form. The stimulus can be added to the tube prior to collecting the biological sample, and the stimulus can be lyophilized. A broad range of compounds are candidate stimuli including small molecules and larger biomolecules such as cytokines, antibodies, and steroids. An example stimulus is 100 nanograms of recombinant human interferon alpha, an immunomodulatory cytokine of known medical importance. Other immunomodulatory cytokines of interest as stimulants include, without limitation, IL-1 and IL-2 (Karupiah et al. (1990) J. Immunology 144:290-298, Weber et al. (1987) J. Exp. Med. 166:1716-1733, Gansbacher et al. (1990) J. Exp. Med. 172:1217-1224, and U.S. Pat. No. 4,738,927); IL-3 and IL-4 (Tepper et al. (1989) Cell 57:503-512, Golumbek et al. (1991) Science 254:713-716, and U.S. Pat. No. 5,017,691); IL-5 and IL-6 (Brakenhof et al. (1987) J. Immunol. 139:4116-4121, and International Publication No. WO 90/06370); IL-7 (U.S. Pat. No. 4,965,195); IL-8, IL-9, IL-10, IL-11, IL-12, and IL-13 (Cytokine Bulletin, Summer 1994); IL-14 and IL-15; alpha interferon (Pinter et al. (1991) Drugs 42:749-765, U.S. Pat. Nos. 4,892,743 and 4,966,843, International Publication No. WO 85/02862, Nagata et al. (1980) Nature 284:316-320, Familletti et al. (1981) Methods in Enz. 78:387-394, Twu et al. (1989) Proc. Natl. Acad. Sci. USA 86:2046-2050, and Faktor et al. (1990) Oncogene 5:867-872); beta-interferon (Seif et al. (1991) J. Virol. 65:664-671); gamma-interferons (Radford et al. (1991) The American Society of Hepatology 20082015, Watanabe et al. (1989) Proc. Natl. Acad. Sci. USA 86:9456-9460, Gansbacher et al. (1990) Cancer Research 50:7820-7825, Maio et al. (1989) Can. Immunol. Immunother. 30:34-42, and U.S. Pat. Nos. 4,762,791 and 4,727,138); G-CSF (U.S. Pat. Nos. 4,999,291 and 4,810,643); Granulocyte Macrophage Colony Stimulating Factor (GMCSF) (International Publication No. WO 85/04188).

Immunomodulatory compounds may also include compounds that are agonists of a Toll-like receptor (TLR). “TLR” generally refers to any Toll-like receptor of any species of organism. Several human TLRs are disclosed in PCT publication no. WO 98/50547. Agonists of human TLRs are also described in Table 1 of Ulevich R, (2004) Nature Reviews: Immunology, 4:512-520; in Table 1 of Akira and Takeda (2004) Nature Reviews Immunology 4:499-511; in Medzhitov R, (2001) Nature Reviews Immunology 1:345-145; and in PCT publication nos. WO 03/031573 and WO 03/103586. Each of the preceding disclosures are incorporated herein by reference.

Many dried or lyophilized compounds such as interferon alpha are extremely soluble in blood and the masses used in the current configuration of the device will dissolve immediately into the blood sample upon contact. Additional components may be added to improve drying down of the stimulus and or solubility in the patient sample including serum albumin and or dextrose. The stimulating and stabilizing agents may be provided in different forms or formulations in the device. By way of illustration, the agent can be admixed with conventional carriers and excipients (i.e., vehicles) and used in the form of powders, aqueous solutions, dispersions, bead dispersions (e.g., where the agent, such as stimuli and/or anticoagulant, is dried onto beads and/or impregnating a soluble bead matrix (1 micron diameter or smaller beads dried down in a highly soluble substrate) to enhance the solubility and consistency of dispersion of certain stimulatory and/or anti coagulation agents), gels, foams, tablets, capsules, elixirs, suspensions, syrups, wafers, and the like. A fluid or liquid composition will generally consist of a suspension, dispersion or solution of the active agent in a suitable liquid carrier(s), for example, water, ethanol, glycerine, sorbitol, non-aqueous solvent such as polyethylene glycol, oils or water, with a suspending agent, preservative, surfactant, wetting agent, or coloring agent. Alternatively, a liquid formulation can be prepared from a reconstitutable powder. For example, a powder containing active compound and a suspending agent can be reconstituted with water to form a suspension or dispersion. Accordingly, there area wide variety of suitable stimulating and stabilizing formulations of the present invention.

In the subject device, an effective amount of a stimulating agent is provided in the first chamber, and an effective amount of a stabilizing agent is provided in the second chamber. By “effective amount” is intended to mean a sufficient amount of the compound to provide the desired utility. For instance, for intracellular signaling or gene induction, the effective amount for the stimulating agent is the amount which elicits or is calibrated to elicit a useful response compared to controls (e.g., increase or decrease in phosphorylated protein content, increase or decrease in post-translational modification of specific proteins, increased mRNA abundance for a gene etc.). An effective amount for the stabilization agent can also be ascertained in this way, for instance, by determining the stability of a desired species in the samples relative to a control. As such, an appropriate effective amount may be determined by one of ordinary skill in the art using only routine experimentation.

The user can then invert the tube several times to ensure proper mixing of the anticoagulant and stimulus with the patient sample. Inverting the tube to ensure proper mixing is a common practice for existing blood draw devices. After 15 minutes, or other desired stimulation time, in one embodiment, the user grasps the tube in both hands and bends it approximately 45 degrees to break the ampoule 103 containing the stabilization liquid releasing the stabilization liquid into the patient sample. The polyethylene body of the tube can be flexible and durable enough to withstand bending without failure and has been used in other applications requiring the breaking of internal ampoules, including Cyalume Lightsticks™. The user then can then invert the tube 10 times to ensure proper mixing of the stabilization liquid with the patient sample.

In another aspect, the second chamber contains at least one stabilizing agent. A stabilizing agent, as referred to herein, can include any agent which maintains in state, i.e., inhibits any further change in, the status of any biomolecule in the biological sample. Such changes can include, without limitation, gene expression; protein expression; nucleic acid abundance such as transcript abundance; nucleic acid degradation; protein or polypeptide abundance/degradation; posttranscriptional modifications to polynucleotides such as, for example, polyadenylation and methylation; spliceosomal association with nucleic acids; any intracellular signalling, such as, e.g., Jak/STAT pathway signalling; nucleic acid hairpin loop and secondary structure formation; posttranslational modifications of polypeptides, such as, without limitation, phosphorylation, methylation, ubiquitination, SUMOylation, heme or prosthetic group coordination; protein conformation; protein binding state, and others as known in the art. Examples of suitable stabilizing agents for stabilizing and preserving nucleic acids and/or preventing gene induction include cationic compounds, detergents, chaotropic salts, ribonuclease inhibitors, chelating agents, and mixtures thereof. A suitable ribonuclease inhibitor is placental RNAse inhibitor protein. Examples of chaotropic salts include urea, formaldehyde, guanidinium isothiocyanate, guanidinium hydrochloride, formamide, dimethylsulfoxide, ethylene glycol and tetrafluoroacetate. The stabilizing agent can also include another component for treating the biological sample. For example, chemical agents can be included to permeabilize or lyse viruses and cells. Other components include proteinases, phenol, phenol/chloroform mixtures, alcohols, aldehydes, ketones and organic acids. The detergents can be anionic detergents, cationic detergents or nonionic detergents. The anionic detergent can be, for example, sodium dodecyl sulfate. Nonionic detergents can be, for example, ethylene oxide condensation products, such as ethoxylated fatty acid esters of polyhydric alcohols. A nonionic detergent of particular interest is a polyoxyethylene sorbitan monolaurate sold under the trade name TWEEN 20 by Sigma Chemical Co. The detergents can be included in an effective amount to permeabilize or to lyse the cells so as to form micelles and other complexes with the nucleic acids.

In some embodiments of the presently disclosed devices and methods, stabilization buffers can contain fixatives and/or precipitants. Fixatives and precipitants are well known in the art and can be readily selected by the skilled artisan based upon the desired assay. Cross-linking fixatives include, without limitation, formaldehyde, glutaraldehyde, paraformaldehyde, ethyldimethyl-aminopropyl-carbodiimide, and dimethyl-silserimidate. Precipitants include ethanol, acetic acid, methanol, acetone, and combinations thereof. Glacial acetic acid can also be included as a fixative. Fixatives are typically of use at concentrations which do not destroy the ability of the cell's nucleic acids or proteins to bind to a probe, depending upon the binding event of interest. Other useful fixatives will be obvious to one skilled in the art. In some embodiments, the concentration of formaldehyde in the stabilization buffer is at least about 0.1%, sometimes about 0.5% sometimes about 0.7%, often about 1%, frequently about 3%, often about 4%, as much as 5%, up to 10% formaldehyde.

RNA stabilization for later analysis of the abundance of RNA transcripts by microarray, polymerase chain reaction or other method: There are multiple chemistries available that can be divided into those that lyse all cells in the sample and those chemistries that stabilize nucleic acids without lysing leukocytes. An example of the first group is Trizol and other buffers that contain phenol and or other organic solvents. An example of the second chemistry is RNALater and other buffers that contain very high concentrations of salt including halide salts like ammonium chloride, but no organic solvents. These buffers and equivalent formulations will be recognized by those experienced in the art.

For assays where hybridization of probes to nucleic acids in cells is desired, an assay solution may typically comprise a chaotropic denaturing agent, a buffer, a pore forming agent, a hybrid stabilizing agent. Chaotropic denaturing agents (Robinson, D. W. and Grant, M. E. (1966) J. Biol. Chem. 241: 4030; Hamaguchi, K. and Geiduscheck E. P. (1962) J. Am. Chem. Soc. 84: 1329) include formamide, urea, thiocyanate, guanidine, trichloroacetate, tetramethylamine, perchlorate, and sodium iodide. Any buffer which maintains pH at least between 7.0 and 8.0 may be utilized. A pore forming agent is for instance, a detergent such as Brij 35, Brij 58, sodium dodecyl sulfate, CHAPS™ TRITON X-100™. Depending on the location of the target biopolymer, the pore-forming agent is chosen to facilitate probe entry through plasma, or nuclear membranes or cellular compartmental structures. For instance, 0.05% Brij 35 or 0.1% TRITON X-100™ will permit probe entry through the plasma membrane but not the nuclear membrane. Alternatively, sodium desoxycholate will allow probes to traverse the nuclear membrane. Thus, in order to restrict binding to cytoplasmic biopolymer targets, nuclear membrane pore-forming agents are avoided. Such selective subcellular localization contributes to the specificity and sensitivity of the assay by eliminating probe binding to complementary nuclear sequences or antigens when the target biopolymer is located in the cytoplasm. Agents other than detergents such as fixatives may also serve this function.

The stabilizing agent is generally selected based on a preference to carry out proteomic or genomic analysis of a treated sample post-stimulation/stabilization.

For instance, the interest in developing stimulation assays as diagnostics or for research purposes is broadly separated into those that wish to focus on the biology of the sample at the protein level (proteomics: intracellular flow cytometry, Western blots etc.) and those that wish to focus on the biology of the sample at the nucleic acid level (genomics: microarrays, PCR etc.). The device and methods of the invention can be applied to meet both needs. Specifically, the device and method may employ different stabilization solutions such as one that stabilizes proteins and intracellular signaling, or one that stabilizes nucleic acid species. For proteomic applications, the stabilizing agent is one that stabilizes proteins and intracellular signaling. Of particular interest are stabilizing agents that lyse erythrocytes but not leukocytes and preserve cell surface antigens while arresting at least one cellular process selected from protein synthesis, protein degradation, RNA synthesis, DNA synthesis, nucleic acid degradation, endocytosis, secretion, phosphorylation, dephosphorylation, ubiquitinization, methylation, and combinations thereof.

Of interest in certain embodiments are stabilizing agents that preserve cell surfaces suitable to permit single-cell sorting, such as fluorescence-activated cell sorting (FACS) or flow cytometry. Of interest is where intracellular phospho-specific antibody staining of the sample is analyzed by flow cytometry or FACS. FACS technology facilitates single-cell multiparametric analysis and sorting, based on physical properties of cells and/or their relative expression levels of specific protein or glycoprotein epitopes and metabolites. The use of fluorescent antibodies specific for unique phosphorylated epitopes—or “phospho-epitopes”—on proteins of interest has further extended the range of FACS analyses. This new application, dubbed “phospho-FACS”, has become a tool of choice for delineating intracellular phosphorylation cascades. As such, the application of phospho-FACS to cellular subsets from blood or the periphery, whether frequent or rare, aids in the discovery of pathological biomarkers and therapeutic innovation. Because of its ability to generate single-cell data and resolve the heterogeneous mixtures of cells present in patient samples, the phospho-FACS technique features numerous advantages compared to other analytical methods for measuring signaling cascades.

It has further been found that samples treated in the above manner suitable for flow cytometry can be analyzed by virtually any proteomic technique, including Western blotting, capillary electrophoresis, microfluidics, mass spectrometry (following purification), inductively coupled plasma mass spectrometry (ICP-MS), and combinations thereof.

As such, in some embodiments the stabilization liquid can be a buffer for subsequent analysis of protein abundance and or post-translational modification of proteins by phospho-specific flow cytometry or other methods requiring single-cell suspensions rather than cell lysates. One effective formulation of stabilization liquid for biological samples including whole blood is a solution of paraformaldehyde in phosphate buffered saline. Introduction of paraformaldehyde into a blood sample to a final concentration between about 0.1% and about 4% can effectively arrest protein degradation and preserve the post-translational modification of proteins involved in intracellular signaling including phosphorylation. Other additives that can be added to the stabilization liquid include diethylene glycol, Triton X100, and or Saponin. In embodiments in which cells obtained in whole blood, such as e.g. immune cells, are to be stimulated and assayed, it may be of interest to lyse erythrocytes in the sample by using an erythrocyte-specific lysis buffer. These additives can improve the ability to stabilize intracellular protein modification states and or lysis of cells. For stimulation assays in which single-cell sorting or flow-cytometric analysis will be required, stabilizing agents can include 0.1%-10% paraformaldehyde. The inventor has also found that including diethylene glycol improves the ability to stabilize intracellular protein modification states and/or lysis of erythrocytes in the sample. In some embodiments, diethylene glycol in is of use in the stabilization agent at final concentrations as low as around 0.001%, sometimes at around 1%, sometimes around 3%, up to about 10% by volume. Also of interest as an ingredient in stabilizing agents is the polar, aprotic organic solvent dimethyl sulfoxide (DMSO). In some embodiments, DMSO in is of use in the stabilization agent at final concentrations of around 1%, up to about 10% by volume. 2,4-dinitrobenzene sulfonic acid sodium salt (DNBS) is also of use at concentrations of about 5-50 mM or around 20-30%. As discussed, detergent TWEEN 20 is of use along with other detergents for the permeabilization of cells to labeled probe. The inventor found that, for the purposes of FACS analysis, the use of TWEEN 20 is preferable over that of either saponin or Triton x100 for the reason that the latter detergents, which contain benzene rings and their delocalized electron systems, result in higher background autofluorescence during analysis.

Preferred embodiments of the stabilizing agent for embodiments involving single-cell sorting or flow-cytometric analysis include aqueous solutions containing final concentrations in the biological sample of: about 0.1%-10% formaldehyde with about 0.001%-10% diethylene glycol; about 0.1%-5% formaldehyde with about 1%-10% dimethyl sulfoxide (DMSO), 5-50 mM 2,4-dinitrobenzene sulfonic acid sodium salt (DNBS) and about 0.001%-1.0% Tween 20; about 1%-3% formaldehyde with about 1%-3% diethylene glycol; and about 0.7%-1% formaldehyde, with 6%-7% DMSO, 20%-30% DNBS and 0.07%-0.2% Tween 20.

Optimal stabilization liquids for proteomics can be prepared using the following steps. Stabilization liquid can be prepared using double distilled H2O (or phosphate buffered saline). Stabilization liquid can be delivered such that the final concentrations in biological samples is 3% formaldehyde and 3% diethylene glycol. Concentrations of these reagents in ampoules may be up to 3× concentration. Alternative formulations of the stabilization liquid can be used that halt synthesis and degradation of nucleic acids in the specimen. Other stabilization liquid formulations that can be used with the invention are known in the art, including those disclosed in US Application 2006/0105372 A1, U.S. Pat. No. 6,204,375 and U.S. Pat. No. 5,346,994, which are herein incorporated by reference in their entirety.

In some embodiments, the processing step of the tube can include analysis by phospho-specific flow cytometry.

In some embodiments of the invention described herein, the ampoule of the tube can have a total volume of about 2 milliliters of stabilization liquid composed of about 4.5% paraformaldehyde, about 4.5% diethylene glycol in double distilled H2O (or phosphate buffered saline). This stabilization liquid can be used to effectively stabilize 2 milliliters of blood in the tube. The stabilizing liquid can also be used for analyzing cytokine-induced post-translational modification of signaling proteins in multiple leukocyte populations in blood drawn from healthy human donors including T cells, B cells, monocytes, and granulocytes.

Additionally, a buffer can be added to the stabilization fluid for subsequent analysis of protein abundance and or post-translational modification of proteins by Western blotting, antibody arrays, protein arrays, or other methods requiring cell lysates. One appropriate formulation is the sodium dodecyl sulfate (SDS) cell lysis buffer normally used for sodium dodecyl sulfate polyacrylamide gel electrophoresis (SDS-PAGE) including broad acting protease inhibitors and phosphatase inhibitors, as known to those skilled in the art.

In some embodiments, the stabilization liquid can cause RNA stabilization for later analysis of the abundance of RNA transcripts by microarray, polymerase chain reaction (PCR) realtime PCR, oligonucleotide microarrays, cDNA microarrays, macroarrays, especially for the purpose of quantifying transcript abundance. There are multiple chemistries available that can be divided into those that lyse all cells in the sample and those chemistries that stabilize nucleic acids without lysing leukocytes. Examples of chemistries that stabilize nucleic acids include but are not limited to, Trizol, and other buffers that contain phenol and/or other organic solvents, or other buffers like RNALater that contain very high concentrations of salt including halide salts like ammonium chloride, but no organic solvents.

After the process is complete, the tube can then transferred to a container filled with dry ice for shipment to a facility for analysis. Alternatively, the tube can be stored in a freezer, ideally one that maintains a temperature at or below −80 degrees Celsius. Storage at −80 degrees Celsius may be adequate to preserve clinically important features of the patient sample including protein modifications and or gene transcript abundance for more than a month, but the effects of storage must be determined in advance.

### II. SYSTEMS

Also provided herein are systems for collecting, assaying and stabilizing a biological sample. In one aspect, the systems include a collection apparatus as described above and additionally, an automation apparatus, also referred to herein as a base station, which automates certain aspects of using the collection apparatus and can facilitate the use of multiple collection apparati in parallel to stimulate, stabilize, and store multiple biological samples, as well as to store and track information regarding each use.

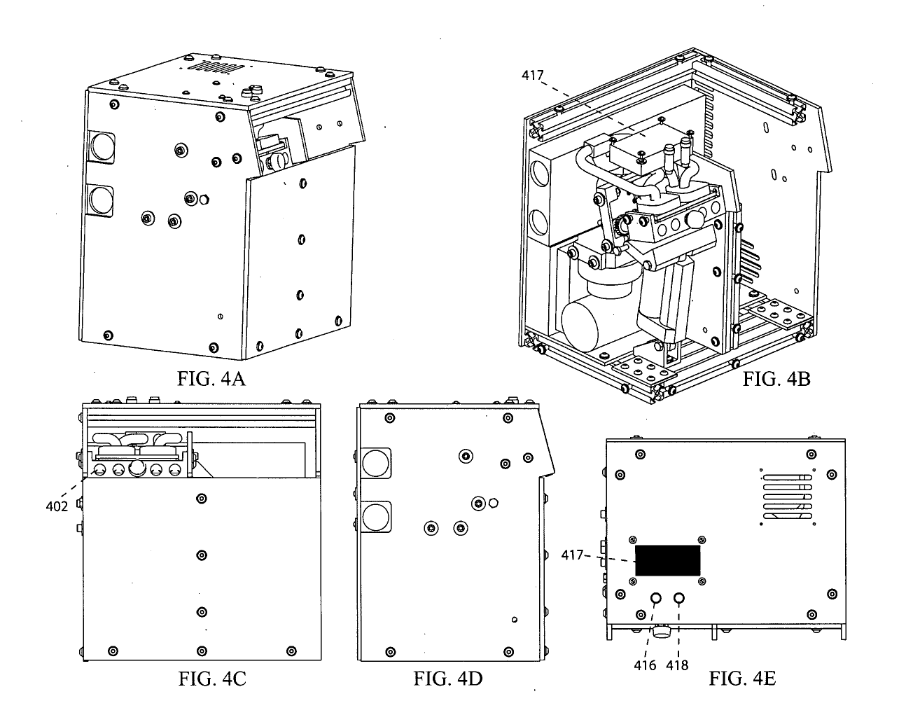

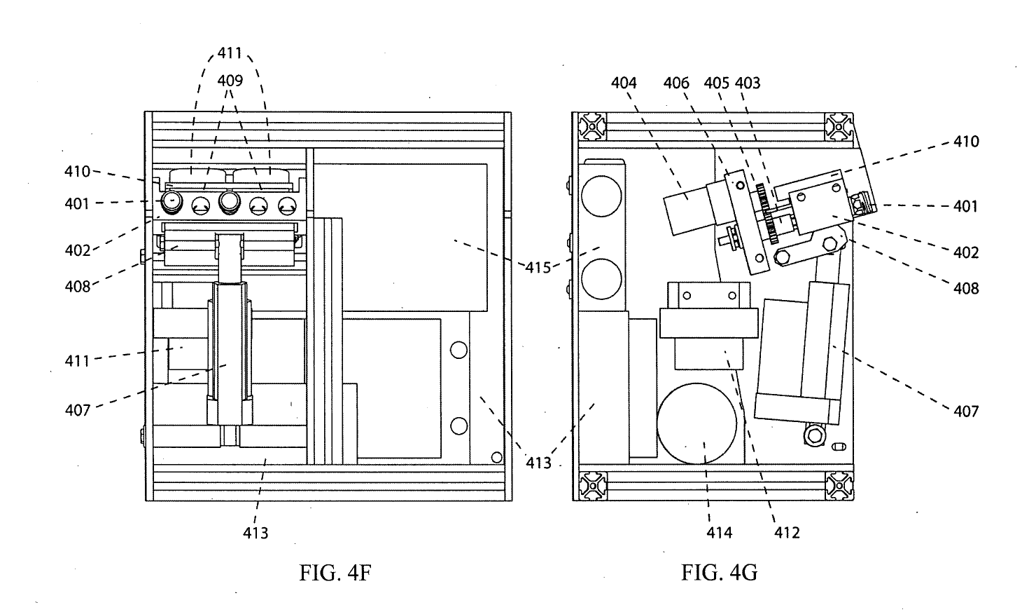

FIG. 4A illustrates one embodiment of a base station. FIG. 4B shows a perspective view of the apparatus in FIG. 4A with the top, left, and front panels removed along with the two top frame members. FIG. 4C illustrates a front view of one embodiment of the apparatus; FIG. 4D illustrates a left side view of one embodiment of the apparatus; FIG. 4E illustrates a top view of one embodiment of the apparatus. FIG. 4F illustrates a front view of one embodiment of the apparatus with the front panel removed. FIG. 4G illustrates a left side view of one embodiment of the apparatus with the left and front panels removed.

The device 401 (tube) can be inserted into complementary holes in a temperature controlled aluminum block 402. The device 401 can pass out of the backside of the block 402 and can insert with a tight fit into the coupling 403. Axial rotation of the coupling 403 can generate axial rotation of the tube by means of the tight, complementary fit between the tube 401 and the coupling 403. A gear motor 404 can rotate the couplings 403 by means of a system of gears 5 that are held in alignment with the holes in the block 402 by means of an alignment plate 406. At the appropriate time an electric linear actuator 407 can press the wedge-shaped armature 408 into the tubes 401 held in the block 402 thereby flexing the walls of the tube 401 and breaking the crushable ampoules inside the tube 401. After the ampoules have been broken, the linear actuator 407 can retract the armature 408 so that the tubes 401 can be freely spun by means of the coupling 403, thus ensuring proper mixing between the stabilizer solution released from the ampoules and the sample material in the tube.

Temperature of the block 402 can be controlled by means of two peltiers 409 attached to the surface of block 402 by means of thermal epoxy that has very high thermal conductivity. The side of the peltiers 409 not affixed to the block 402 can be attached to a copper heat spreader 410 by means of thermal epoxy. The copper heat spreader 409 can be attached to two water blocks by means of thermal epoxy. When the peltiers 409 are actively cooling the block 402 they are pumping heat out of the block 402 by means of transferring phonons from the side of the peltier 409 attached to the block 402 to the side attached to heat spreader 410. Waste heat is also generated in this process. The sum of this heat passively diffuses from the heat spreader 410 into chamber 411 where it is conveyed by means of the water-based coolant pumped through chamber 411 by the coolant pump 412 and thereon to the fan cooled radiator 413. From the fan cooled radiator 413 the coolant can return to the main coolant reservoir 414 and from which it is pumped by the coolant pump 412 back through the circuit. The twelve volt power supply 415 provides power for all the components of the base station. To begin a cycle, the user places Smart Tubes into the tube block 402 and presses the Start Button 416. An LCD screen 417 provides status information to the user. At the conclusion of the run the Smart Tubes are cooled to 4-8 degrees Celsius and held at that temperature until the user presses the Stop Button 418 and transfers the tubes to −80 C storage or ships them on dry ice to a laboratory for analysis.

It will be understood by those skilled in the art that any convenient thermal elements may be employed to heat and/or cool the containers and their contents and thus control the temperature of the reaction mixture in the internal compartment. In general, suitable heating elements for heating the block include conductive heaters, convection heaters, or radiation heaters. Examples of conductive heaters include resistive or inductive heating elements coupled to the block, e.g., resistors or thermoelectric devices. Suitable convection heaters include forced air heaters or fluid heat exchangers for flowing fluids past the block. Suitable radiation heaters include infrared or microwave heaters. The heating element may comprise metals, tungsten, polysilicon, or other materials that heat when a voltage difference is applied across the material. Similarly, various cooling elements may be used to cool the block. For example, various convection cooling elements may be employed such as a fan, peltier device, refrigeration device, or jet nozzle for flowing cooling fluids past the surfaces of the block. Alternatively, various conductive cooling elements may be used.

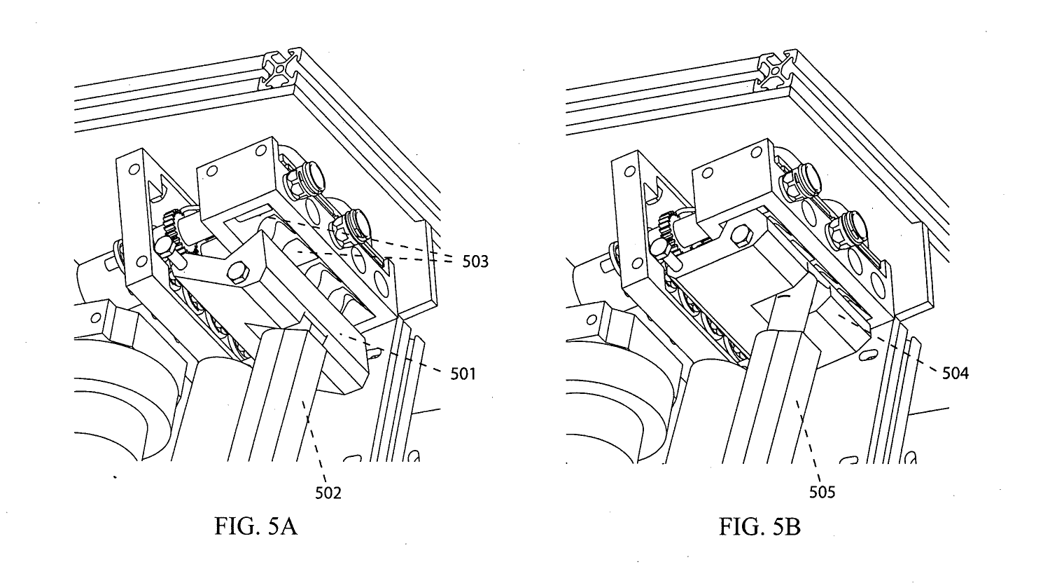

FIG. 5A illustrates the armature of the base station, shown here holding two tubes, in the open position. FIG. 5B illustrates the armature of the base station in the closed position. The armature 501 is in the open position during most of the time the base station is operated. When the linear actuator 502 extends it presses the armature 501 into the flexible walls of the devices 503 (tubes) flexing the walls and breaking the stabilizer ampoules inside the devices. Sample mixing in the tubes is by means of axial rotation of the tubes. The armature is shown in the closed position in 504 and the linear actuator in the extended position in 505.

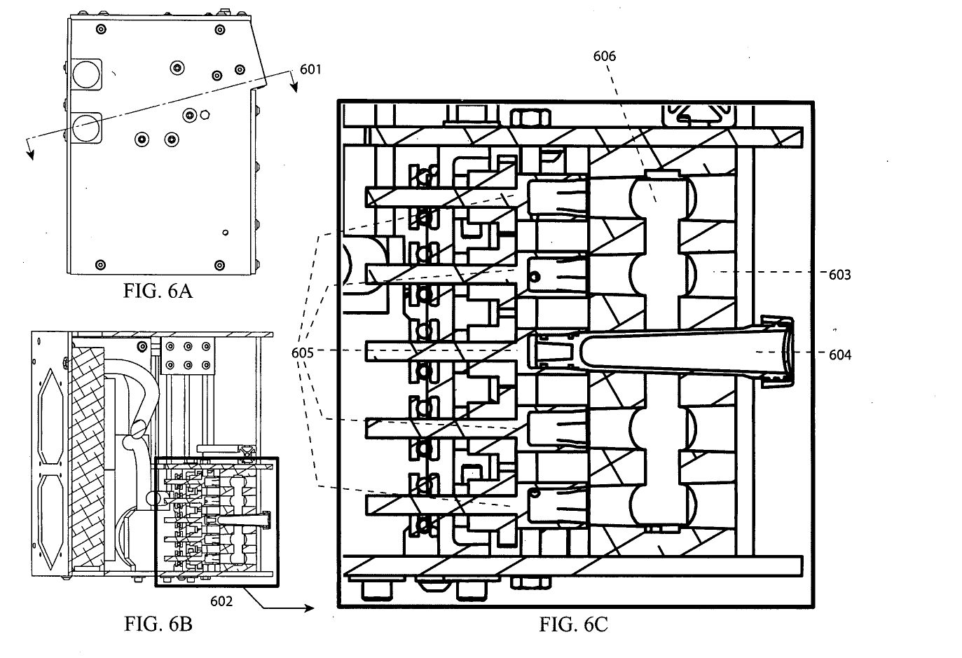

FIG. 6 shows a cross section of the block of the base station including the tube couplings that mate with the distal ends of the tube and transmit axial rotation to the tubes. FIG. 6A the plane of the cross sectional view in FIG. 6B is shown as a dotted line 601. The thick line square 602 shows the region that is enlarged in FIG. 6C. The tapered holes in the tube block 603 are complementary in shape to the device 604, one instance of which is shown inserted in the tube block. The distal end of the device mates with one of the couplings 605 which rotates the device along its long axis. The slot in the tube block 606 provides clearance for the armature to exert a force on the flexible wall of the tubes in a direction perpendicular to the long axis of the tubes.

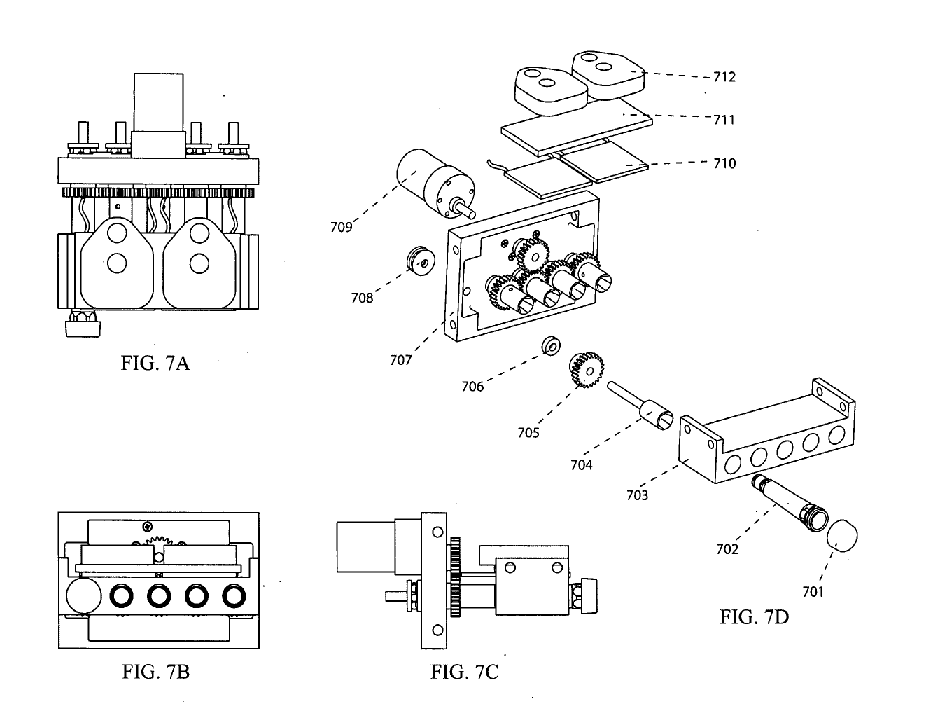

FIG. 7 illustrates the detailed workings of the tube block sub-assembly. FIG. 7A shows a top view of tube block sub-assembly with one tube in it. FIG. 7B shows a front view of the tube block sub-assembly with one tube in it. FIG. 7C shows a left side view of the tube block sub-assembly with one tube. 701 is a tube cap; 702 is a Smart Tube; 703 is the tube block; 704 is the coupling that mates with the bottom of the Smart Tube and transfers axial rotational torque through the complementary interaction of hexagonal faces on the bottom of the Smart Tube (similar to a socket wrench, but the faces are tapered); 705 is the spur gear with a Fairloc Hub that turns the coupling and meshes to other spur gears that translate the rotational motion created by the gear motor 709; 705 has a Fairloc Hub which works as a shaft collar to lock the spur gear to the shaft of 704; 706 is a thrust bearing that allows the subassembly of 704 and 705 to rotate and be supported by the shaft alignment plate 707; 708 is a thrust bearing and shaft collar that allows the subassembly of 704 and 705 to rotate and be supported by 707; The shaft collar locks onto the shaft of 704 to hold the subassembly tightly together while thrust bearings 706 and 708 allow it to rotate; a pair of peltiers 710 thermally control the tube block 703 by pumping heat (phonons) into or out of the heat block; the peltiers are in thermal communication with a pair of water blocks 712 via a copper heat spreader 711;

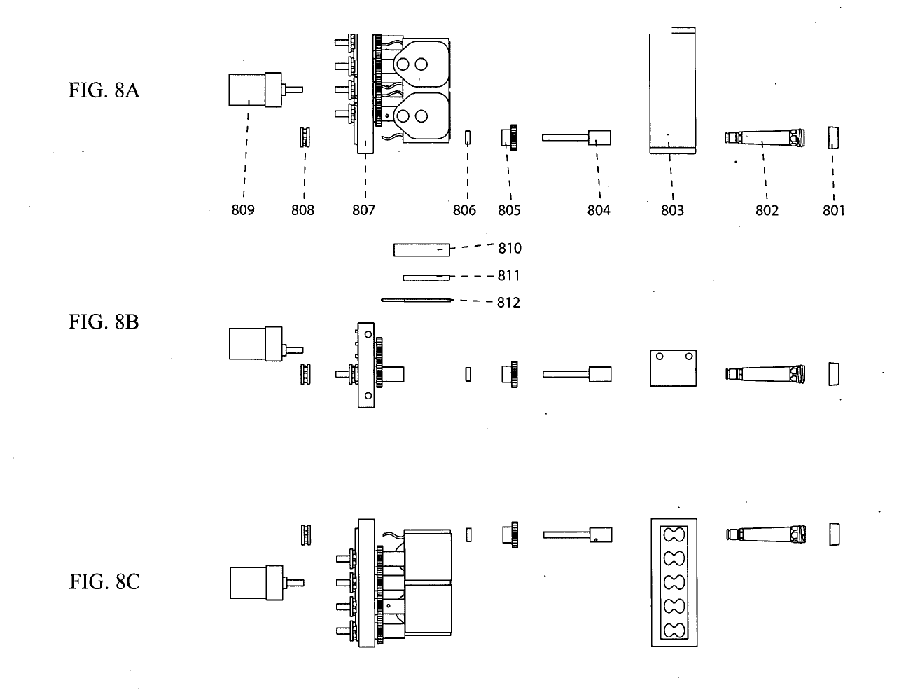

FIG. 8A Exploded top view of the tube block sub-assembly with one tube. 801 is a tube cap; 802 is a Smart Tube; 803 is the tube block; 804 is the coupling that mates with the bottom of the Smart Tube and transfers axial rotational torque through the complementary interaction of hexagonal faces on the bottom of the Smart Tube (similar to a socket wrench, but the faces are tapered); 805 is the spur gear with a Fairloc Hub that turns the coupling and meshes to other spur gears that translate the rotational motion created by the gear motor 809; 805 has a Fairloc Hub which works as a shaft collar to lock the spur gear to the shaft of 804; 806 is a thrust bearing that allows the subassembly of 804 and 805 to rotate and be supported by the shaft alignment plate 807; 808 is a thrust bearing and shaft collar that allows the subassembly of 804 and 805 to rotate and be supported by 807; The shaft collar locks onto the shaft of 804 to hold the subassembly tightly together while thrust bearings 806 and 808 allow it to rotate; a pair of peltiers 810 thermally control the tube block 803 by pumping heat (phonons) into or out of the heat block; the peltiers are in thermal communication with a pair of water blocks 812 via a copper heat spreader 811. FIG. 8B Exploded left view of the tube block sub-assembly with one tube. FIG. 8C Exploded bottom view of the tube block sub-assembly with one tube;

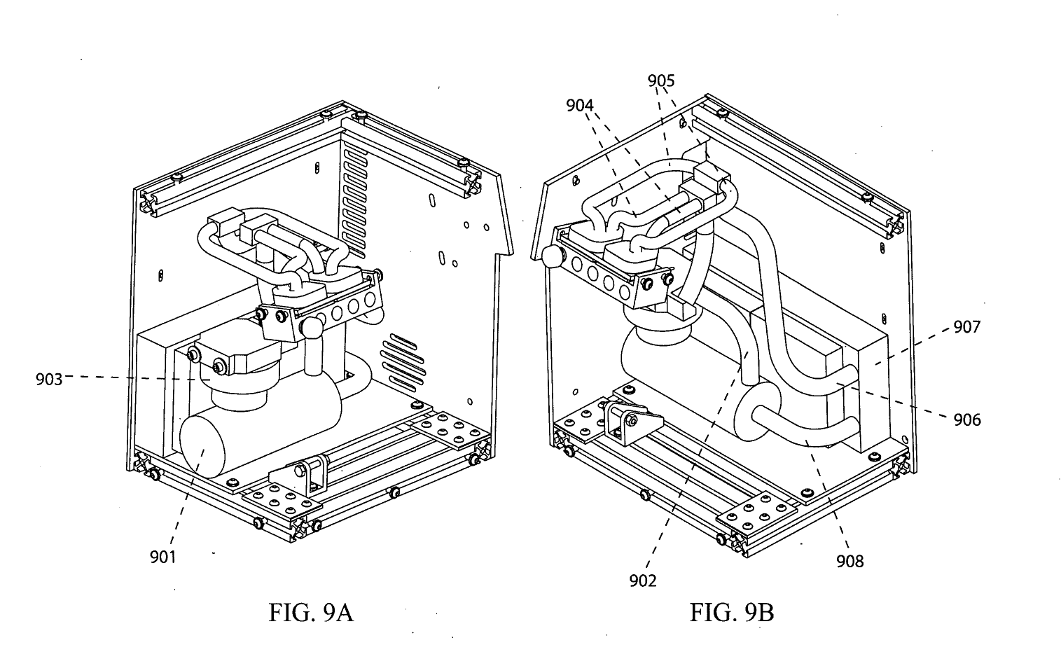

FIG. 9A Perspective view of the tube block and liquid cooling system with other components removed for clarity. Water based coolant is pumped from the reservoir 901 via coolant hose 902 by pump 903. The coolant is then pumped via hoses 904 into water blocks which are maintained at a near-constant temperature by the circulation of the coolant. The coolant exits the water blocks by hoses 905 which unify into hose 906 which carries the coolant into the fan-cooled radiator (also known as a heat exchanger) 907. Coolant hose 908 complete the coolant circuit by returning the coolant to the reservoir 901 from radiator 907. FIG. 9B Perspective view of the tube block and liquid cooling system with other components removed for clarity

### III. METHODS

Methods of using the container apparatus disclosed herein, as well as the automated system, are provided. Methods of collecting, stimulating and stabilizing a biological sample therewith are disclosed. In one aspect the methods include providing a sample collection container including a side wall, a bottom wall, in which at least one wall is constructed of an elastically deformable material, and a closure member defining an internal compartment, the internal compartment having arranged therein a partition defining and fluidly separating first and second chambers in the internal compartment, the first chamber positioned in association with the closure member to receive the biological sample; at least one stimulating agent in the first chamber in an amount effective to stimulate a biological sample; and at least one stabilizing agent in the second chamber in an amount effective to stabilize the biological sample; collecting a biological sample from a patient and introducing the biological sample into the first chamber so as to expose the biological sample to the stimulating agent stimulating the biological sample in the first chamber for a preselected period of time, to produce a stimulated biological sample; and stabilizing the stimulated biological sample after the preselected period of time by compromising the partition and mixing contents of the first and second chambers.

By “preselected period of time” is meant that the biological sample and stimulating agent in the first chamber is admixed with the stabilizing agent in the second chamber anywhere from immediately after to up to 1 hour or more, after the biological sample is received in the first chamber of the device. In general, stimulation of the sample in the first chamber ranges in increments of seconds from about 5 minute to 30 minutes, and usually from about 10 minutes to about 20 minutes, depending on the sample and particular assay of interest.

In some embodiments, the biological sample is collected from the patient directly into the first chamber of the sample collection container. In further embodiments, the biological sample is collected from the patient into a container which is not the sample collection container and is thereafter introduced into the first chamber of the sample collection container.

A biological sample for which the provided devices, methods and kits find use include, by way of example, whole blood.

However, it will be clear to the skilled artisan that the methods disclosed herein have extremely wide applicability. The use of plastic containers with multiple, discrete compartments for the collection, storage, assaying and culturing of cells and tissue in the molecular biological arts is widespread and nearly unlimited in the diversity of cells to which it may be applied. The devices, systems and methods herein disclosed provide a way to render the contents of isolated chambers within a plastic container transitionable to a single chamber, without compromising the integrity or sterility of the container or the rapidity of the assay. As such, the skilled artisan will recognize that the disclosed embodiments of the invention are useful for executing any multistep assay wherein a cell or tissue is serially exposed to at least a stimulus and a stabilizer so as to preserve results of a bioassay for subsequent processing and analysis. As such, any biological sample from any individual or patient may be stimulated and stabilized according to the methods of the present invention.

Accordingly, biological samples for which the provided methods and kits find use may include, without limitation, whole blood, synovial fluid, cerebrospinal fluid, amniotic fluid and tissue biopsies including tumor cells, such as from friable tumors. The biological sample can be a body fluid or solid biopsy obtained from a patient. In one embodiment, the biological sample is whole blood. Other biological samples include cell-containing compositions such as red blood cell concentrates, platelet concentrates, leukocyte concentrates, plasma, serum, urine, bone marrow aspirates, tissue, cells, and other body fluids. Also of interest are solid tissue samples, e.g., easily dissociated biopsies.

Of interest are hematologic disorders. Hematologic disorders include abnormal growth of blood cells which can lead to dysplastic changes in blood cells and hematological malignancies such as various leukemias. Examples of hematological disorders include but are not limited to acute myeloid leukemia, acute promyelocytic leukemia, acute lymphoblastic leukemia, chronic myelogenous leukemia, the myelodysplastic syndromes, and sickle cell anemia.

Other examples of cancers, cells from which may be obtained and analyzed according to the herein disclosed methods include, but are not limited to, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Since any tissue which can be obtained from a patient can readily be exposed to a stimulus and subsequently stabilized according to the disclosed methods, any cellular change resulting from such exposure can be analyzed thereafter.

In a further aspect, the methods disclosed herein include providing an automation apparatus as described above, in which the apparatus holds the sample collection container and its contents at 37 degrees Celsius during a preselected time period for simulating the biological sample and rotates it along its long axis to ensure mixing with the stimuli in the container; then deforms the container wall to compromise the partition; rotates the sample collection container along its long axis to mix contents of the first and second chambers; incubates the sample collection container for a predetermined time at a predetermined temperature during the stabilization; and lowers the temperature of the sample collection container and its contents to between −80 degrees Celsius and 10 degrees Celsius. In some aspects, the method further includes storing and/or shipping the sample at a storage temperature at or below room temperature.

A patient sample can be added to one or more tubes. The patient sample can be added manually by a user of the system. In some embodiments, the patient sample can be added automatically. The tube or tubes can then be inserted into the apparatus. The filled tubes can be added into complementary holes or docks in a thermally controlled aluminum block that is part of a base station. The holes can be pre-heated to physiologic temperature (approximately 37 degrees Celsius). The base station can have a microelectronic means, such as a microprocessor or microcontroller, that controls the functioning of the base station. Once the base station is activated, the automated cycle can begin. Alternatively, the base station can detect which holes or docks are occupied and then the base station can start automatically. When the tube is inserted into the dock, the lower portion of the tube can pass through a hole in the dock and into a coupling that holds the bottom of the tube. The inside diameter of the coupling can be slightly larger than the outside diameter of the bottom of the tube, but smaller than the outside diameter of the rubber O-ring on the tube. When the tube is inserted into the coupling, the rubber O-ring can be slightly deformed thereby creating a snug fit between the tube and the coupling. Intermittently, the tube can be rotated along its long axis by the rotation of the coupling. This rotational torque can be transferred to the tube by the coefficient of static friction between the rubber O-ring of the tube and the coupling. Rotation of the tube ensures that the blood inside the tube mixes completely with the stimuli inside the tube and reaches a uniform temperature throughout. The tube also has hexagonal faces that mate with complementary faces in the coupling—this mating allows greater torque to be applied by the coupling on the tube and ensures axial rotation of the tube by the coupling.

After a defined period of time, the base station can automatically activate the tube. The defined period of time can be measured from the time the base station is activated and can be approximately 15 minutes. The tube can be activated by the steps of the electric motor driven linear actuator driving a wedge-shaped swing armature into the flexible wall of the dock. The driving motion can cause the crushing of the ampoules inside the tube. After the armature activates the tube, the contents can be mixed by the axial rotation discussed previously. After the stabilizer has acted on the blood for a prescribed period of time, for example 10 minutes, the base station can then lower the temperature of the holes or dock to a temperature appropriate for the short term storage of the samples. In some embodiments, the holes or dock are lowered to about 8 degrees Celsius. The base station can then signal that the processing of the tube has finished. In some embodiments, the base station can signal that processing is complete by any sensory device including, but not limited to, illuminating a light emitting diode (LED) (or like element), emitting a tone, or any combination thereof.

Once the processing is complete, the activated tube can remain in the holes of the dock for several hours before the tube has to be transferred to a freezer or a box of dry ice for shipping. Temperature control of the docks can be controlled by microprocessor controlled peltiers that can be attached via thermal epoxy to the aluminum tube block that holds the tube. The peltiers can be attached to the aluminum block with or without additional fittings. In some embodiments only one peltier is attached. In some embodiments, more than one peltier can be attached. The microprocessor can use a sensor, including but not limited to a thermistor, thermocouple, or like device, to determine the current temperature of the dock or holes. The microprocessor can then determine the difference between the current temperature and the desired temperature for that step of the experiment and uses a control feedback algorithm such as a proportional, integrative, derivative (PID) controller to determine how much power to supply the peltier. The microprocessor can then send pulse width modulated (PWM) signals to an H-bridge to supply power to the peltiers. The direction of current flow through the peltiers can dictate whether the peltiers heat or cool the dock or holes. The width of the PWM signals (duty cycle) can determine the average voltage, and thus effective power, that can be sent to the peltiers. The sides of the peltiers not in contact with the tube block can be attached via a heat-spreader to a water block (or air-based heat sink). A liquid can be pumped through the circuit. The circuit can carry the liquid through the water blocks and a fan blown radiator unit. The water blocks and cooling circuit can keep one side of the peltiers near to room temperature. Keeping one side of the peltiers near room temperature allows the peltiers to efficiently heat or cool the dock as required by the experimental protocol.

Some embodiments of the herein disclosed methods further include analyzing the sample by proteomic or genomic methods. Such proteomic or genomic methods include, without limitation, flow cytometry, multilabeled time-of-flight mass spectroscopy, protein microarrays, PCR, real time quantitative PCR, nucleic acid microarrays, RNAi arrays, cell arrays, cDNA microarrays, peptide sequencing, and nucleic acid sequencing.

### IV. KITS

Kits for collecting, assaying, stabilizing, and analyzing a biological sample according to the present methods are also provided. In one aspect, such a kit includes a collection device as described above.

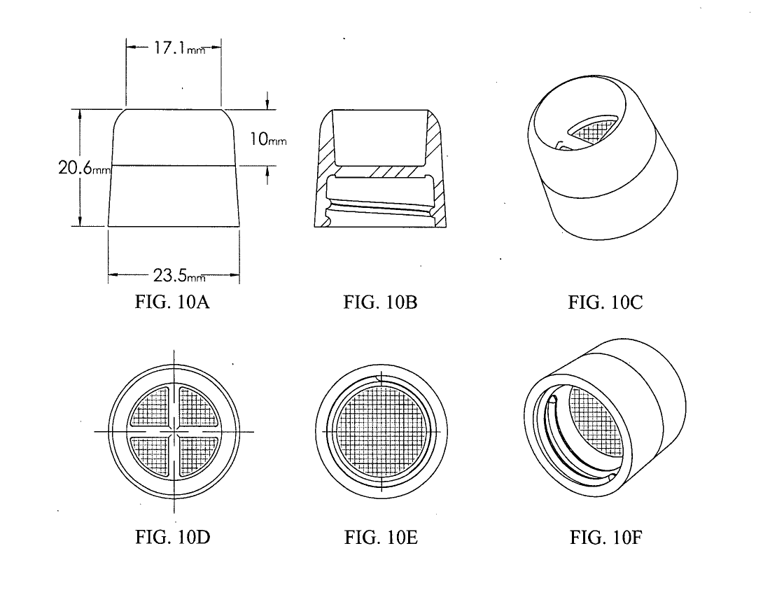

In an additional embodiment, a kit for analyzing and processing a biological sample is provided. In some embodiments the kit contains a filter cap capable of replacing the closure member of the provided container apparatus, the filter cap including a mesh through which liquid can be added or removed to the internal compartment while retaining within the internal compartment fragments of compromised the partition. FIG. 10 shows the filter cap that can be used to keep fragments of the ampoule inside the container (Smart Tube) when the contents are decanted. The cross-hatch pattern shows the nylon filter attached to the filter cap—it is this filter that removes ampoule fragments. The opening size of this mesh is in the range between 250 microns and 2000 microns, such as, e.g., about 500 microns to about 1000 microns. A coarse nylon filter is used is to ensure good flow-through, since finer meshes restrict entry of air and are not conducive to decanting small volumes. The ampoule retention insert shown in FIG. 1 is effective at removing larger ampoule fragments, while this filter cap is effective at removing ampoule fragments of nearly any size. The insert and the cap can be used independently or in combination.

In another aspect, the kit further contains a hypotonic lysis buffer; a hypertonic lysis buffer; a permeabilization buffer; and a staining buffer. After removal from storage, cells can be subjected to osmotic stress by treatment with hypo- and, optionally, hypertonic lysis buffers in series. In some embodiments of the kit, the hypotonic lysis buffer and the hypertonic lysis buffer include detergent. In preferred embodiments, the detergent is Tween 20. In general, the higher the amount of fixative, such as paraformaldehyde, present in the stabilization buffer, the greater the need for detergent in the first (hypotonic) and second (hypertonic) lysis steps in order to lyse unwanted cells, such as erythrocytes in a case where whole blood is the biological sample. Following permeabilization of the cells of interest, the cells may be stained for an antigen of interest, e.g., with labeled antibodies. The use of the reagents and kits disclosed herein is made clear to one of skill in the art by the illustrative examples below.

Biological samples for which the claimed devices, systems, methods and kits may find use include, without limitation, whole blood, synovial fluid, cerebrospinal fluid, amniotic

### V. EXAMPLES

### Example 1

### Analysis of Stabilized Biological Sample Using Phospho-Specific Flow Cytometry

One process for analysis by phospho-specific flow cytometry includes the following steps. Frozen samples can be washed two times with ddH₂O at physiological pH that may include an agent for lysing remaining erythrocytes if the biological sample was blood. Optionally, 0.1% Triton X100 or 0.1% saponin can be added to the ddH2O used to lyse the erythrocytes, detergents that have been shown to be effective for lysing erythrocytes. The cells are then washed with phosphate buffered saline and the pellet resuspended in 2 milliliters of a solution of 80% methanol and 20% phosphate buffered saline chilled to 4 degrees Celsius. The methanol fixed cell suspension can then be stored at −80 degrees Celsius. To continue processing the methanol fixed cell suspension is washed 2 times with staining media consisting of 0.5% bovine serum albumin dissolved in phosphate buffered saline and then stained and analyzed by phospho-FACS, as known in the art.

### Example 2

### Analysis of Stabilized Biological Sample Using the Smart Tube Kit for Processing Samples Frozen in Smart Tubes, with Subsequent Analysis by Phospho-Specific Flow Cytometry

The following protocols uses the described container apparatus and Kit for processing samples frozen in Smart Tubes for subsequent analysis by phospho-specific flow cytometry.

Components of the Processing Kit include:
i. Filter Cap (size of filter mesh openings between 500 microns and 2000 microns)
ii. Lysis Buffer 1: 0.03% Tween 20 in double distilled H2O (ddH2O)
iii. Lysis Buffer 2: 0.03% Tween 20 in 2× phosphate buffered saline (2×PBS)
iv. One Liter of 2×PBS=16 g NaCl, 0.4 g KCl, 2.88 g Na₂HPO₄, 0.48 g of KH₂PO4, and has a pH of 7.4.
v. Permeabilization Buffer 1: 80% methanol with 20% PBS. (pre-chill on ice before use)
vi. Staining Buffer 1: 0.5% bovine serum albumin in PBS

### A. Thawing Collected, Stimulated, Stabilized Whole Blood Samples; Lysing Erythrocytes:

Thaw samples in 37 C water bath for 10 minutes. Unscrew cap, add 2 ml of Lysis Buffer 1, reattach the cap and vortex for 10 seconds. Replace the cap with the Filter Cap and decant into a 15 ml conical tube. Optionally, a 50 ml conical tube can be substituted with a cell strainer in place to remove cell clumps. Top off the conical tube with Lysis Buffer 1 and incubate in a 37 C water bath (42 C is an alternative temp with unique advantages) for 10 minutes. Centrifuge at 800×g for 5 minutes. Discard supernatant. If resulting pellet is white (is free of unlysed erythrocytes) proceed to next section, Staining For Analysis By Phospho-Specific Flow Cytometry. If resulting pellet is red (has unlysed erythrocytes) resuspend the pellet in 10 ml of Lysis Buffer 2 and incubate in 37 C water bath (42 C is an alternative temp with unique advantages) for 10 minutes. Centrifuge the tubes at 800×g for 5 minutes, decant, and wash the pellet with Lysis Buffer 1. The resulting pellet should be white, corresponding to complete erythrocyte lysis. Proceed to section on staining for analysis by phospho-specific flow cytometry.

### B. Staining for Analysis by Phospho-Specific Flow Cytometry:

Vortex the tubes containing the pellets from the above to loosen the pellets. Add 1 ml of Permeabilization Buffer 1 to each tube and vortex for 5 seconds to resuspend the pellet in the buffer. Transfer the tubes to −80 C. The samples can be stored at −80 C for at least 30 days before further processing. For further processing, add at least 4 ml of Staining Buffer to each tube and centrifuge at 800×g for 5 minutes at 4 C. Decant and wash the pellet two more times with 4 ml washes of Staining Buffer. Resuspend each pellet in 100 ul of Staining Buffer and transfer 100 ul of the cell suspension to a new FACS tube (or plate) for antibody staining. Add the antibody cocktail to each sample and stain in the dark for 30 minutes at room temperature. Antibody cocktails for this application typically include one or more fluorescently labeled phospho-specific antibodies such as clone 47 specific for STAT5 (pY694) (Becton Dickinson catalog number 612598) and one or more fluorescently labeled antibodies specific for cell-type restricted surface epitopes such as clone P67.6 specific for CD33 (Becton Dickinson catalog number 341640). Top off the tubes with Staining Buffer, centrifuge at 800×g and decant. Analyze on the appropriate flow cytometry platform for the application, such as the Becton Dickinson FACSCalibur or LSRII. Note that if 1 ml of patient blood was collected and stimulated in the Smart Tube, as recommended, then the sample can be split into at least 4 different FACS tubes for analysis with different staining cocktails.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

What is claimed is:

1. An apparatus for collecting, assaying and stabilizing a biological sample, said apparatus comprising:
a container having a side wall, a bottom wall, and a closure member defining an internal compartment, said internal compartment having arranged therein a partition defining and fluidly separating first and second chambers in said internal compartment, said first chamber positioned in association with said closure member to receive said biological sample;
wherein at least one said wall is constructed of an elastically deformable material;
wherein said first chamber contains at least one stimulating agent;
wherein said second chamber contains at least one stabilizing agent; and
wherein said first and second chambers can be placed in fluid communication by deforming said at least one wall without opening or otherwise compromising the fluid integrity of said internal compartment.

2. The apparatus of claim 1, wherein said partition is constructed of a material the fluid integrity of which can be compromised by deformation of said wall so as to place said first and second chambers in fluid communication.

3. The apparatus of claim 2, wherein said partition is constructed of a material that is one or more of: breakable and dissolvable.

4. The apparatus of claim 2 further comprising one of: a mesh or aperture;
through which liquid can be added or removed to said internal compartment while retaining within said internal compartment fragments of compromised said partition.

5. The apparatus of claim 3, wherein said partition forms an ampoule defining said second chamber.

6. The apparatus of claim 5, wherein said ampoule is constructed of borosilicate glass.

7. The apparatus of claim 1, wherein said wall further comprises a support ring at the interior of said internal compartment;
wherein said partition comprises a disc member affixed by a breakable adhesive to said support ring, said affixed disc member defining and fluidly separating said first and second chambers in said internal compartment;
wherein said disc member is constructed of a material substantially less elastically deformable than said wall, such that said disc member can be displaced by deformation of said wall and support ring, so as to place in fluid communication said first and second chambers.

8. The apparatus of claim 7, wherein said support ring is an integral support ring and is at a non-normal angle relative to the long axis of said apparatus.

9. The apparatus of claim 8, wherein said angle is about 45 degrees.

10. The apparatus of claim 1 further comprising an anticoagulant agent present in said first chamber.

11. The apparatus of claim 1, wherein said stimulating agent is a biological agent.

12. The apparatus of claim 11, wherein said stimulating agent is an antibody.

13. The apparatus of claim 11, wherein said stimulating agent is a small molecule.

14. The apparatus of claim 11, wherein said stimulating agent is a cytokine.

15. The apparatus of claim 14, wherein said stimulating agent is an immunomodulatory cytokine.

16. The apparatus of claim 11, wherein said stimulating agent is a Toll-Like Receptor ligand.

17. The apparatus of claim 1, wherein said stabilizing agent comprises a fixative.

18. The apparatus of claim 1, wherein said stabilizing agent comprises a cell lysis buffer.

19. The apparatus of claim 1, wherein said stabilizing agent maintains cell surface antigens while arresting at least one cellular process selected from the group consisting of: protein synthesis, protein degradation, nucleic acid synthesis, nucleic acid degradation, endocytosis, secretion, phosphorylation, dephosphorylation, ubiquitinization, and methylation.

20. The apparatus of claim 1, wherein said stabilizing agent preserves nucleic acids.

21. The apparatus of claim 1, wherein said stabilizing agent contains a cell lysis buffer or erythrocyte specific cell lysis buffer.

22. The apparatus of claim 1, wherein said stabilizing agent comprises a fixative and a erythrocyte lysis buffer.

23. The apparatus of claim 1, wherein said stabilizing agent stabilizes proteins and intracellular signaling.

24. The apparatus of claim 1 wherein said stabilizing agent preserves nucleic acids for subsequent analysis by polymerase chain reaction (PCR), realtime PCR, oligonucleotide microarrays, cDNA microarrays, macroarrays, especially for the purpose of quantifying transcript abundance.

25. The apparatus of claim 1, wherein said stabilizing agent preserves cell surfaces suitably to permit single-cell sorting and or flow cytometric analysis.

26. The apparatus of claim 25, wherein said single-cell sorting is fluorescence-activated cell sorting.

27. The apparatus of claim 26, wherein said fluorescence-activated cell sorting and or flow cytometric analysis utilizes phospho-specific antibodies.

28. The apparatus of claim 22, wherein said stabilizing agent is an aqueous solution comprising a final concentration in said biological sample of about 0.1%-10% formaldehyde, 0.001%-10% diethylene glycol.

29. The apparatus of claim 22, wherein said stabilizing agent is an aqueous solution comprising a final concentration in said biological sample of about 0.1%-5% formaldehyde, 1%-10% dimethyl sulfoxide (DMSO), 5-50 mM 2,4-dinitrobenzene sulfonic acid sodium salt (DNBS), 0.001%-1.0% Tween 20 detergent.

30. The apparatus of claim 22, wherein said stabilizing agent is an aqueous solution comprising a final concentration in said biological sample of 1%-3% formaldehyde and 1%-3% diethylene glycol.

31. The apparatus of claim 22, wherein said stabilizing agent is an aqueous solution comprising a final concentration in said biological sample of 0.7%-1% formaldehyde, 6%-7% DMSO, 20%-30% DNBS, 0.07%-0.2% Tween 20 detergent.

32. The apparatus of claim 1, wherein said first chamber has an internal pressure that is lower than atmospheric pressure.

33. The apparatus of claim 32, wherein said internal pressure is specified to draw a predetermined volume of said biological sample into said first chamber.

34. The apparatus of claim 1, wherein said biological sample is chosen from: whole blood, synovial fluid, cerebrospinal fluid, amniotic fluid and tumor cells.

35. A system for collecting, assaying and stabilizing a biological sample, said system comprising:
a) a collection apparatus according to claim 1; and
b) an automation apparatus comprising:
i) a manipulation means capable of manipulating said collection apparatus by one or more of: moving, rotating, shaking, ultrasonically vibrating and subsonically vibrating said collection apparatus;
ii) a force-exerting means capable of placing in fluid communication said first and second chambers of said collection apparatus; and
iii) a thermal regulation means capable of regulating the temperature of said collection apparatus.

36. The system according to claim 35 further comprising a microelectronic element controlling the functions of said automation apparatus.

37. The system according to claim 35 further comprising a user interface capable of reporting the status of the system to a user.

38. The system according to claim 36 further comprising a timing means in functional communication with, and arranged so as to trigger the operation of one or more of: said manupulation means, said force-exerting means and said thermal regulation means.

39. The system according to claim 36 wherein said collection apparatus further comprises a unique tag allowing its identification.

40. The system according to claim 36 wherein said unique tag is selected from the group consisting of: an RFID tag, a linear bar code, a matrix bar code, and a microdot pattern.

41. The system according to claim 39 wherein said automation apparatus collects data comprising assay parameter data for one or more tagged collection apparati.

42. The automation apparatus according to claim 41 further comprising a means of transmitting said assay parameter data to a remote location.

43. The automation apparatus according to claim 42 wherein said remote location is an external processing system capable of one or more of: storing said data, analyzing said data and displaying said data to a user.

44. The system according to claim 35, wherein:
said partition is constructed of a material the fluid integrity of which can be compromised by deformation of said wall so as to place said first and second chambers in fluid communication; and
said force-exerting means is capable of placing in fluid communication said first and second chambers of said collection apparatus by deforming said wall.

45. The system according to claim 35, wherein said wall further comprises a support ring at the interior of said internal compartment;
wherein said partition comprises a disc member affixed by a breakable adhesive to said support ring, said affixed disc member defining and fluidly separating said first and second chambers in said internal compartment;
wherein said disc member is constructed of a material substantially less elastically deformable than said wall such that said disc member can be displaced by deformation of said wall and support ring, so as to place in fluid communication said first and second chambers.

46. A method of collecting, stimulating and stabilizing a biological sample, said method comprising:
providing a sample collection container comprising a wall constructed of an elastically deformable material, a bottom wall, and a closure member defining an internal compartment, said internal compartment having arranged therein a partition defining and fluidly separating first and second chambers in said internal compartment, said first chamber positioned in association with said closure member to receive said biological sample; at least one stimulating agent in said first chamber in an amount effective to stimulate a biological sample; and at least one stabilizing agent in said second chamber in an amount effective to stabilize said biological sample;
collecting a biological sample from a patient and introducing said biological sample into said first chamber so as to expose said biological sample to said stimulating agent;
stimulating said biological sample in said first chamber for a preselected period of time, to produce a stimulated biological sample; and
stabilizing said stimulated biological sample after said preselected period of time by compromising said partition and mixing contents of said first and second chambers to produce a stabilized biological sample.

47. The method according to claim 46, wherein said stabilizing agent contains a cell lysis buffer or erythrocyte specific cell lysis buffer.

48. The method according to claim 46, wherein said stabilizing agent comprises a fixative and an erythrocyte lysis buffer.

49. The method according to claim 46, wherein said stabilizing agent stabilizes proteins and intracellular signaling.

50. The method according to claim 49, wherein said stabilized biological sample is subsequently analyzed by a proteomic technique.

51. The method according to claim 50, wherein said proteomic technique is a Western blotting technique.

52. The method according to claim 50, wherein said proteomic technique is a capillary electrophoretic technique.

53. The method according to claim 50, wherein said proteomic technique is a microfluidic technique.

54. The method according to claim 46 wherein said stabilizing agent preserves nucleic acids for subsequent analysis by one or more of: a polymerase chain reaction (PCR), realtime PCR, oligonucleotide microarray, cDNA microarrays and macroarray technique.

55. The method according to claim 54 wherein said technique is a sequencing technique.

56. The method according to claim 46, wherein said stabilizing agent preserves cell surfaces suitable to permit single-cell sorting and/or flow cytometric analysis.

57. The method according to claim 56, wherein said single-cell sorting is fluorescence-activated cell sorting.

58. The method according to claim 57, wherein said fluorescence-activated cell sorting utilizes phospho-specific antibodies.

59. The method according to claim 56, wherein said flow cytometric analysis is inductively coupled plasma mass spectrometry (ICP-MS).

60. The method according to claim 46, wherein said partition is constructed of a material the fluid integrity of which can be compromised by deformation of said wall so as to place said first and second chambers in fluid communication; and
wherein said compromising said partition is accomplished by deforming said wall.

61. The method according to claim 46 further comprising: providing an automation apparatus according to claim 35, wherein said apparatus:
holds said sample collection container and its contents at 37 degrees Celsius during said simulating;
deforms said wall to compromise said partition;
rotates said sample collection container along its long axis to mix contents of said first and second chambers;
incubates said sample collection container for a predetermined time at a predetermined temperature during said stabilization; and
lowers the temperature of said sample collection container and its contents to between negative 80 degrees Celsius and 10 degrees Celsius.

62. The method according to claim 46, wherein said biological sample is collected from said patient directly into said first chamber of said sample collection container.

63. The method according to claim 46, wherein said biological sample is collected from said patient into a container which is not said sample collection container and is thereafter introduced into said first chamber of said sample collection container.

64. A method of collecting, stimulating and stabilizing a whole blood sample, said method comprising:
providing a sample collection container having a side wall, a bottom wall, and a closure member defining an internal compartment, said internal compartment having arranged therein: (i) a partition defining and fluidly separating first and second chambers in said internal compartment, said first chamber positioned in association with said closure member to receive said biological sample and said first chamber having pressure less than atmospheric pressure; and (ii) at least one stimulating agent contained within said first chamber in an amount effective to stimulate a whole blood sample, and at least one stabilizing agent contained within said second chamber in an amount effective to stabilize said whole blood sample; wherein at least one said wall is constructed of an elastically deformable material;
collecting a whole blood sample directly from a patient into said first chamber so as to immediately expose said whole blood sample to said stimulating agent;
stimulating said whole blood sample in said first chamber for a desired period of time so as to form a stimulated whole blood sample; and
stabilizing said stimulated whole blood sample immediately after said desired period of time by compromising said partition by deforming said wall and mixing contents of said first and second chambers.

65. The method according to claim 46 wherein said stimulating further comprises maintaining said sample at predetermined reaction temperature for a predetermined period of time.

66. The method according to claim 46, said method further comprising one or more of: storing said sample at a storage temperature at or below room temperature and shipping said sample at a storage temperature at or below room temperature.

67. The method according to claim to 46, said method further comprising analyzing said sample by proteomic or genomic methods.

68. The method according to claim 67, wherein said proteomic or genomic methods are chosen from flow cytometry, protein microarrays, PCR, real time quantitative PCR, nucleic acid microarrays, RNAi arrays, cell arrays, cDNA microarrays, peptide sequencing, and nucleic acid sequencing.

69. A method of analyzing a stimulation profile of a biological sample, said method comprising analyzing a biological sample for a stimulation profile, said biological sample prepared by a method according to any claim 46.

70. A method according to claim 46, said method further comprising processing the sample by heating it to a temperature between room temperature and 100° C.

71. A method according to claim 46, said method further comprising processing the sample by heating it to a temperature between 40° C. and 50° C.

72. A kit for collecting, assaying and stabilizing a biological sample, said kit comprising an apparatus according to any claim 1.

73. A kit for analyzing and processing a biological sample, said kit comprising:
a filter cap capable of replacing the closure member of an apparatus according to claim 3,
said filter cap comprising a mesh or aperture through which liquid can be added or removed to said internal compartment while retaining within said internal compartment fragments of compromised said partition;
a hypotonic lysis buffer;
a hypertonic lysis buffer;
a permeabilization buffer; and
a staining buffer.

74. The kit according to claim 73, wherein openings in said mesh are greater than about 500 microns in size and less than about 2000 microns in size.

75. The kit according to claim 73, wherein one or more of said hypotonic lysis buffer and said hypertonic lysis buffer comprises detergent.

76. The kit according to claim 75, wherein said detergent is Tween 20.
